(19) United States
(12) Patent Application Publication
(10) Pub. No.: US 2005/0113328 A1
(43) Pub. Date: May 26, 2005

(54) Method and antisense compound for potentiating anti-cancer agents

(76) Inventors: Gayathri R. Devi, Morrisville, NC (US); Patrick L. Iversen, Corvallis, OR (US)

Correspondence Address: PERKINS COIE LLP, P.O. BOX 2168, MENLO PARK, CA, 94026, US

(21) Appl. No.: 10/981,989

(22) Filed: Nov. 4, 2004

Related U.S. Application Data
(60) Provisional application No. 60/518,139, filed on Nov. 6, 2003.

(51) Int. Cl. A61K 48/00; A61K 31/675; C07F 9/6533

(52) U.S. Cl. 514/44; 514/81; 544/81

(57) ABSTRACT

A method and compound for enhancing the lethality of an anti-cancer therapy, such as radiation, chemotherapy, or TRAIL protein, are disclosed. The compound is composed of morpholino subunits joined by phosphorodiamidate linkages, and has a targeting sequence that is complementary to an AUG start, IRES, or splice-donor region of the transcript for human X-linked inhibitor of apoptosis protein (XIAP). The method includes exposing cancer cells to the compound.

This patent application claims priority to U.S. provisional patent application Ser. No. 60/518,139 filed on Nov. 6, 2003, which is hereby incorporated herein in its entirety by reference.

### FIELD OF THE INVENTION

This invention relates to methods for potentiating the antitumor activity of anticancer agents, including radiation, small-molecule chemotherapeutic drugs, and the TRAIL peptide, in cancer cells, particularly in cancer cells that have become resistant to the agent.

### REFERENCE

The following references are related to the background of the invention and/or may be related to certain protocols or methods useful in making or using the invention.

Agrawal, S., S. H. Mayrand, et al. (1990). “Site-specific excision from RNA by RNase H and mixed-phosphate-backbone oligodeoxynucleotides.” Proc Natl Acad Sci USA 87(4): 1401-5.

Anderson, K. P., M. C. Fox, et al. (1996). “Inhibition of human cytomegalovirus immediate-early gene expression by an antisense oligonucleotide complementary to immediate-early RNA.” Antimicrob Agents Chemother 40(9): 2004-11.

Bennett, M. R. and S. M. Schwartz (1995). “Antisense therapy for angioplasty restenosis. Some critical considerations.” Circulation 92(7): 1981-93.

Blumenreich, M. S., T. M. Woodcock, et al. (1985). “High-dose cisplatin in patients with advanced malignancies.” Cancer 55(5): 1118-22.

Bonham, M. A., S. Brown, et al. (1995). “An assessment of the antisense properties of RNase H-competent and steric-blocking oligomers.” Nucleic Acids Res 23(7): 1197-203.

Boudvillain, M., M. Guerin, et al. (1997). “Transplatin-modified oligo(2′-O-methyl ribonucleotide)s: a new tool for selective modulation of gene expression.” Biochemistry 36(10): 2925-31.

Byhardt, R. W. (1995). “Turning up the heat on nonsmall cell lung cancer: is the toxicity of concurrent cisplatin-based chemotherapy and accelerated fractionation acceptable?” Int J Radiat Oncol Biol Phys 31(2): 431-3.

Devi, G. R., J. R. Oldenkamp, et al. (2002). “Inhibition of human chorionic gonadotropin beta-subunit modulates the mitogenic effect of c-myc in human prostate cancer cells.” Prostate 53(3): 200-10.

Ding, D., S. M. Grayaznov, et al. (1996). “An oligodeoxyribonucleotide N3′→P5′ phosphoramidate duplex forms an A-type helix in solution.” Nucleic Acids Res 24(2): 354-60.

Forastiere, A. A., T. Leong, et al. (2001). “Phase III comparison of high-dose paclitaxel+cisplatin+granulocyte colony-stimulating factor versus low-dose paclitaxel+cisplatin in advanced head and neck cancer: Eastern Cooperative Oncology Group Study E1393.” J Clin Oncol 19(4): 1088-95.

Gandara, D. R., E. A. Perez, et al. (1991). “Cisplatin chemoprotection and rescue: pharmacologic modulation of toxicity.” Semin Oncol 18(1 Suppl 3): 49-55.

Gee, J. E., I. Robbins, et al. (1998). “Assessment of high-affinity hybridization, RNase H cleavage, and covalent linkage in translation arrest by antisense oligonucleotides.” Antisense Nucleic Acid Drug Dev 8(2): 103-11.

Holcik, M., H. Gibson, et al. (2001). “XIAP: apoptotic brake and promising therapeutic target.” Apoptosis 6(4): 253-61.

Hudziak, R. M., E. Barofsky, et al. (1996). “Resistance of morpholino phosphorodiamidate oligomers to enzymatic degradation.” Antisense Nucleic Acid Drug Dev 6(4): 267-72.

Hudziak, R. M., J. Summerton, et al. (2000). “Antiproliferative effects of steric blocking phosphorodiamidate morpholino antisense agents directed against c-myc.” Antisense Nucleic Acid Drug Dev 10(3): 163-76.

Jones, D. P. and R. W. Chesney (1995). “Renal toxicity of cancer chemotherapeutic agents in children: ifosfamide and cisplatin.” Curr Olin Pediatr 7(2): 208-13.

Lappalainen, K., A. Urtti, et al. (1994). “Cationic liposomes improve stability and intracellular delivery of antisense oligonucleotides into CaSki cells.” Biochim Biophys Acta 1196(2): 201-8.

Lou, X., K. L. Garrett, et al. (2001). “Synthetic hydrogels as carriers in antisense therapy: preliminary evaluation of an oligodeoxynucleotide covalent conjugate with a copolymer of 1-vinyl-2-pyrrolidinone and 2-hydroxyethyl methacrylate.” J Biomater Appl 15(4): 307-20.

Miyake, H., M. Pollak, et al. (2000). “Castration-induced up-regulation of insulin-like growth factor binding protein-5 potentiates insulin-like growth factor-I activity and accelerates progression to androgen independence in prostate cancer models.” Cancer Res 60(11): 3058-64.

Nagase, M., T. Nomura, et al. (1987). “Effects of intralesional versus ip administration of cisplatin on squamous cell carcinoma of mice.” Cancer Treat Rep 71(9): 825-9.

Nicolaou, K. C., Z. Yang, et al. (1994). “Total synthesis of taxol.” Nature 367(6464): 630-4.

Onoda, J. M., K. K. Nelson, et al. (1988). “Cisplatin and nifedipine: synergistic antitumor effects against an inherently cisplatin-resistant tumor.” Cancer Lett 40(1): 39-47.

Pari, G. S., A. K. Field, et al. (1995). “Potent antiviral activity of an antisense oligonucleotide complementary to the intron-exon boundary of human cytomegalovirus genes UL36 and UL37.” Antimicrob Agents Chemother 39(5): 1157-61.

Peters, G. J., C. L. van der Wilt, et al. (2000). “Basis for effective combination cancer chemotherapy with antimetabolites.” Pharmacol Ther 87(2-3): 227-53.

Srivastava, R. K. (2001). “TRAIL/Apo-2L: mechanisms and clinical applications in cancer.” Neoplasia 3(6): 535-46.

Stein, D., E. Foster, et al. (1997). “A specificity comparison of four antisense types: morpholino, 2′-O-methyl RNA, DNA, and phosphorothioate DNA.” Antisense Nucleic Acid Drug Dev 7(3): 151-7.

Summerton, J. and D. Weller (1997). “Morpholino antisense oligomers: design, preparation, and properties.” Antisense Nucleic Acid Drug Dev 7(3): 187-95.

Theon, A. P., J. R. Pascoe, et al. (1993). “Intratumoral chemotherapy with cisplatin in oily emulsion in horses.” J Am Vet Med Assoc 202(2): 261-7.

Toulme, J. J., R. L. Tinevez, et al. (1996). “Targeting RNA structures by antisense oligonucleotides.” Biochimie 78(7): 663-73.

Williams, A. S., J. P. Camilleri, et al. (1996). “A single intra-articular injection of liposomally conjugated methotrexate suppresses joint inflammation in rat antigen-induced arthritis.” Br J Rheumatol 35(8): 719-24.

Zhivotovsky, B., B. Joseph, et al. (1999). “Tumor radiosensitivity and apoptosis.” Exp Cell Res 248(1):10-7.

### BACKGROUND OF THE INVENTION

The National Cancer Institute estimates 1,334,100 new cancer cases are expected to be diagnosed in the United States during 2003 and 556,500 people will die from the disease. Prostate and lung cancers are the leading cause of death in men while breast cancer and lung cancer are the leading cause of death in women. It is also estimated that 8.9 million Americans with a history of cancer were alive in 1999. Worldwide the mortality statistics are more dismal due, in part, to the lack of early diagnosis opportunities available in developing countries.

Despite advances in cancer treatment strategies, lack of efficacy and/or significant side effects due to the toxicity of currently used chemotherapeutic agents remains a problem. Drug toxicity can be severe enough to result in life threatening situations requiring administration of drugs to counteract side effects, and may result in the reduction or discontinuation of the chemotherapeutic agent. One of the major limitations to clinical use of cancer therapeutic agents is the development of resistance to the treatment. The problem of drug resistance has been observed with a number of chemotherapeutic agents. Such resistance is typically evidenced by recurrence of the tumor subsequent to chemotherapy. These drawbacks to current therapies impact negatively on the patient's treatment and quality of life.

Given the extensive side effects and lack of long term efficacy of current chemotherapeutic treatment regimens, new or improved cancer treatment regimens that reduce or eliminate such side effects and/or exhibit enhanced therapeutic efficacy would be of significant value to the medical community.

### SUMMARY OF THE INVENTION

In one aspect, the invention includes a method of enhancing the lethality of an anti-cancer agent selected from radiation or a small-molecule chemotherapeutic compound in mammalian cancer cells. Before, during or after exposing the cells to the anti-cancer agent, the cells are exposed to an antisense compound in an amount effective to enhance the lethality/dose of the cells to the agent. The compound is characterized by (i) 12-40 morpholino subunits, (ii) a substantially uncharged, phosphorus-containing backbone linking a morpholino nitrogen of one subunit to a 5′ exocyclic carbon of an adjacent subunit, (iii) active uptake by mammalian cancer cells, and (iv) a base sequence that is complementary to a target region containing at least 12 contiguous bases in a preprocessed or processed human X-linked inhibitor of apoptosis protein (XIAP) and which includes at least 6 contiguous bases of the sequence selected from the group consisting of: SEQ ID NOS: 7-12. In addition, the compound is capable of hybridizing with a processed or preprocessed XIAP transcript to form a heteroduplex structure having a Tm of dissociation of at least 45° C.

In one preferred embodiment, the morpholino subunits in the antisense compound are joined by phosphorodiamidate linkages, in accordance with the structure: 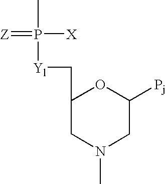
where Y₁═O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino, for example where X═NR₂, where each R is independently hydrogen or methyl.

In one embodiment, the base sequence of the antisense compound is be targeted against the start site of the processed XIAP transcript, and may include at least 6 contiguous bases of one of the sequences identified as SEQ ID NOS:7-9, e.g. Exemplary antisense sequences include one of SEQ ID NOS:7-9. In another embodiment, the base sequence of the antisense compound is targeted against the IRES site of the processed XIAP transcript, and may include at least 6 contiguous bases of the sequence identified as SEQ ID NO:10. An exemplary antisense sequence includes the sequence identified as SEQ ID NO: 10.

In still another embodiment, the base sequence of the antisense compound is targeted against a donor or acceptor splice site of the preprocessed XIAP transcript, and may include has at least 6 contiguous bases of the sequences identified as SEQ ID NOS: 11 and 12. Exemplary antisense sequences include those identified as SEQ ID NOS:11 or 12.

Prior to exposing the cells to the antisense compound, the treated cells may be identified as showing diminished responsiveness to the agent, as evidenced by diminished lethality per dose toxic agent over time.

For use in treating a cancer in a mammalian subject, the exposing step may include administering the antisense compound to the subject before, during, or after treating the subject with the anti-cancer agent. The antisense compound and agent are preferably administered alternately to the subject, at intervals separated by 1-5 days. An exemplary chemotherapeutic agent is cis-platin or an analog thereof. The method may be applied, for example, in treating an androgen-independent prostate cancer.

In another aspect, the invention includes treating a cancer in a mammalian subject, by administering to the subject an antisense compound targeted against the transcript for human X-linked inhibitor of apoptosis protein (XIAP), and the TRAIL protein. The antisense compound is characterized by: (i) 12-40 subunits, (ii) a substantially uncharged, phosphorus-containing backbone linking said subunits, (iii) active uptake by mammalian cells, and (iv) a base sequence that is complementary to a target region containing at least 12 contiguous bases in a preprocessed or processed human X-linked inhibitor of apoptosis protein (XIAP) and which includes at least 6 contiguous bases of the sequence selected from the group consisting of: SEQ ID NOS: 7-12. The compound is capable of hybridizing with a processed or preprocessed human X-linked inhibitor of apoptosis protein (XIAP) transcript to form a heteroduplex structure having a Tm of dissociation of at least 45° C. The amount of oligonucleotide analog administered is effective to inhibit XIAP levels in the subject's cancer cells; the amount of TRAIL administered is effective to inhibit growth of the cancer cells, and the extent of inhibition of growth of the cancer cells is greater than in the absence of step(a).

The antisense compound may have the structural and base-sequence features discussed above. The method may be used, for example, in treating an androgen-independent prostate cancer. The characterization of the cancer as androgen-independent may be determined by (a) administering said antisense compound to the subject, (b) at a selected time later, obtaining a sample of a body fluid from the subject; and assaying the sample for the presence of a nuclease-resistant heteroduplex composed of the antisense compound complexed with a complementary-sequence portion of or preprocessed or processed XIAP transcript.

In still another aspect, the invention provides an oligonucleotide analog compound for use in treating cancer in a subject. The compound is characterized by: (i) 12-40 morpholino subunits, (ii) a substantially uncharged, phosphorus-containing backbone linking a morpholino nitrogen of one subunit to a 5′ exocyclic carbon of an adjacent subunit, (iii) active uptake by mammalian cancer cells, and (iv) a base sequence that is complementary to a target region containing at least 12 contiguous bases in a preprocessed or processed human X-linked inhibitor of apoptosis protein (XIAP) and which includes at least 6 contiguous bases of the sequence selected from the group consisting of: SEQ ID NOS: 7-12. The compound is capable of hybridizing with a processed or preprocessed human X-linked inhibitor of apoptosis protein (XIAP) transcript to form a heteroduplex structure having a Tm of dissociation of at least 45° C. Various structural and base-sequence embodiments of the compound are as described above.

These and other objects and features of the invention will become more fully apparent when the following detailed description is read in conjunction with the accompanying figures and examples.

### BRIEF DESCRIPTION OF THE FIGURES

FIG. 1 shows several preferred morpholino-type subunits having 5-atom (A), six-atom (B) and seven-atom (C-D) linking groups suitable for forming polymers;

FIGS. 2A-D show the repeating subunit segment of exemplary morpholino oligonucleotides, designated A through D, constructed using subunits A-D, respectively, of FIG. 1.

FIGS. 3A-3G show examples of uncharged linkage types in oligonucleotide analogs.

FIGS. 4A-C depict the effect of cisplatin on DU145 human prostate cancer cells. FIGS. 4A and B show cell viability after treatment with cisplatin for 24 h or with indicated concentrations of cisplatin at four different time points, respectively. FIG. 4C represents an immunoblot analysis of lysates from cells treated with cisplatin at 48 and 96 h time points. XIAP and caspases were probed with anti-XIAP, anti-caspase-3 and anti-caspase-7 monoclonal antibodies. The arrows point to the inactive (35 kDa), intermediate (32 kDa) and active (20 kDa) forms of caspase-7.

FIGS. 5A-C depict the effect of TRAIL on cell viability and protein expression in DU145 human prostate cancer cells. FIGS. 5A and B show cell viability after treatment with TRAIL for 24 h or with indicated concentrations of TRAIL at four different time points, respectively. FIG. 5C represents an immunoblot analysis of lysates from cells treated with TRAIL for 6 h. XIAP, caspase-3 and Akt were probed with anti-XIAP (monoclonal), anti-caspase-3 (monoclonal) and anti-Akt (polyclonal) antibodies. The arrows point to the 57 kDa XIAP, active form of caspase-3, and 60 kDa Akt bands.

FIGS. 6A-C represent the effect of XIAP antisense PMO on XIAP expression and cell proliferation in DU145 cells. FIG. 6A are the results from a plasmid-based, luciferase assay system for screening PMO sequence specificity and antisense activity in transfected HeLa cells. FIG. 6B is an immunoblot analysis of lysates from cells treated with XIAP antisense PMO. XIAP expression was determined by probing lysates with anti-XIAP monoclonal antibody. The arrows indicate the 57 kDa XIAP and 43 kDa β-actin control bands. FIG. 6C are representative phase-contrast photomicrographs of DU145 cells scrape loaded from different treatments as indicated.

FIGS. 7A-C depict the effect of XIAP antisense PMO on caspase-3 activation and Akt levels in DU145 cells. FIG. 7A represents an immunoblot analysis of lysates from cells treated with XIAP antisense and scrambled PMOs by scrape loading for 24 h. Caspase-3 activation was monitored by probing lysates with anti-caspase-3 monoclonal antibody. The arrows point to the p17 active form of caspase-3 and 43 kDa β-actin bands. FIG. 7B shows the levels of M30-antigen in cell lysates as determined by ELISA-based method. FIG. 7C represents an immunoblot analysis of lysates from cells treated with XIAP antisense and scrambled PMOs by scrape loading for 24 h. Akt levels were determined by probing lysates with anti-Akt polyclonal antibody. The arrows indicate the 60 kDa Akt and 43 kDa β-actin control bands.

FIGS. 8A-C illustrate the role of combined XIAP antisense PMO and cisplatin treatment on protein expression and cell proliferation in DU145 human prostate cancer cells. Cell viability was determined after treatment with cisplatin for 24 h followed by a 24-h XIAP antisense PMO or control PMO (FIG. 8A). FIG. 8B depicts an immunoblot analysis of XIAP expression after treatment with cisplatin or cisplatin combined with XIAP antisense PMO. The arrows indicate the 57 kDa XIAP and 43 kDa β-actin control bands. FIG. 8C is an immunoblot analysis of caspase-3 activation after the same treatments. The arrows indicate the 32 kDa inactive form of caspase-3 and 43 kDa β-actin control bands.

FIGS. 9A-C depict the effect of combined TRAIL and XIAP antisense PMO on cell proliferation and protein expression in DU145 cells. Cell viability was determined after treatment with XIAP antisense and scrambled PMOs for 24-h followed by a 24-h TRAIL treatment (FIG. 9A). FIG. 9B represents an immunoblot analysis of XIAP expression after the combined treatment compared to controls. The arrows point to the 57 kDa XIAP and 43 kDa β-actin control bands. FIG. 9C depicts an immunobot analysis of Akt levels from cells treated as in FIG. 9B by probing lysates with anti-Akt polyclonal antibody. Immunoblots shown in FIGS. 4-9 were stripped and probed with antibody to β-actin as loading controls.

FIGS. 10A-C depict the effect of ionizing radiation on cell viability (FIG. 10A) at 0-7 days post-treatment and XIAP expression levels (FIG. 10B) at 1, 4 and 7 days post-treatment. FIG. 10C shows that cell viability is reduced in the presence of XIAP antisense PMO in combination with 10 Gy of ionizing radiation.

### DETAILED DESCRIPTION OF THE INVENTION

I. Definitions

The terms below, as used herein, have the following meanings, unless indicated otherwise.

As used herein, the terms “antisense compound”, “antisense agent”, “antisense oligomer” and “antisense oligonucleotide analog” are used interchangeably with respect to the antisense oligonucleotides of the invention. Similarly, the terms “compound” and “agent” may be used interchangeably with respect to the chemotherapeutic compounds for use in practicing the invention.

As used herein, the terms “antisense oligonucleotide” and “antisense oligomer” are used interchangeably and refer to a sequence of nucleotide bases and a subunit-to-subunit backbone that allows the antisense oligomer to hybridize to a target sequence in an RNA by Watson-Crick base pairing, to form an RNA: oligomer heteroduplex within the target sequence. The oligomer may have exact sequence complementarity to the target sequence or near complementarity. Such antisense oligomers may block or inhibit translation of the mRNA containing the target sequence, or inhibit gene transcription, may bind to double-stranded or single stranded sequences, and may be said to be “directed to” a sequence with which it hybridizes.

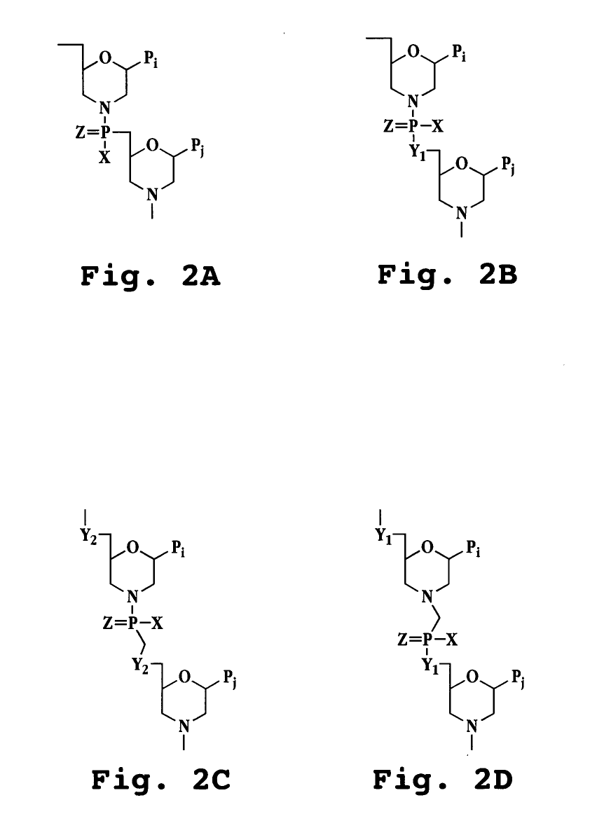

As used herein, a “morpholino oligomer” refers to a polymeric molecule having a backbone which supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. A preferred “morpholino” oligonucleotide is composed of morpholino subunit structures of the form shown in FIG. 2B, where (i) the structures are linked together by phosphorous-containing linkages, one to three atoms long, joining the morpholino nitrogen of one subunit to the 5′ exocyclic carbon of an adjacent subunit, and (ii) Pᵢ and Pⱼ are purine or pyrimidine base-pairing moieties effective to bind, by base-specific hydrogen bonding, to a base in a polynucleotide.

This preferred aspect of the invention is illustrated in FIG. 2B, which shows two such subunits joined by a phosphorodiamidate linkage. Morpholino oligonucleotides (including antisense oligomers) are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

As used herein, a “nuclease-resistant” oligomeric molecule (oligomer) is one whose backbone is not susceptible to nuclease cleavage of a phosphodiester bond. Exemplary nuclease resistant antisense oligomers are oligonucleotide analogs, such as phosphorothioate and phosphate-amine DNA (pnDNA), both of which have a charged backbone, and methyl-phosphonate, morpholino, and peptide nucleic acid (PNA) oligonucleotides, all of which may have uncharged backbones.

As used herein, an oligonucleotide or antisense oligomer “specifically hybridizes” to a target polynucleotide if the oligomer hybridizes to the target under physiological conditions, with a thermal melting point (Tₘ) substantially greater than 37° C., preferably at least 50° C., and typically 60° C.-80° C. or higher. Such hybridization preferably corresponds to stringent hybridization conditions, selected to be about 10° C., and preferably about 5° C. lower than the Tₘ for the specific sequence at a defined ionic strength and pH. At a given ionic strength and pH, the Tₘ is the temperature at which 50% of a target sequence hybridizes to a complementary polynucleotide.

Polynucleotides are described as “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree that one polynucleotide is complementary with another) is quantifiable in terms of the proportion of bases in opposing strands that are expected to form hydrogen bonds with each other, according to generally accepted base-pairing rules.

As used herein the term “analog” with reference to an oligomer means a substance possessing both structural and chemical properties similar to those of a reference oligomer.

As used herein, a first sequence is an “antisense sequence” with respect to a second sequence if a polynucleotide whose sequence is the first sequence specifically binds to, or specifically hybridizes with, the second polynucleotide sequence under physiological conditions.

As used herein, a “base-specific intracellular binding event involving a target RNA” refers to the sequence specific binding of an oligomer to a target RNA sequence inside a cell. For example, a single-stranded polynucleotide can specifically bind to a single-stranded polynucleotide that is complementary in sequence.

As used herein, “nuclease-resistant heteroduplex” refers to a heteroduplex formed by the binding of an antisense oligomer to its complementary target, which is resistant to in vivo degradation by ubiquitous intracellular and extracellular nucleases.

As used herein, “XIAP ” refers to X-linked inhibitor of apoptosis. “XIAP” has been associated with regulation of apoptosis and functions as an antiapoptotic protein in various types of cancers, as further detailed below.

As used herein, the term “XIAP antisense oligomer” refers to a nuclease-resistant antisense oligomer having high affinity (ie, which “specifically hybridizes”) to a complementary or near-complementary XIAP nucleic acid sequence, in particular, a processed or preprocessed XIAP mRNA.

As used herein, “TRAIL” refers to tumor necrosis factor (TNF)-related apoptosis-inducing ligand (also known as Apo2 ligand) which preferentially induces apoptotic death in a variety of cancer cells but not normal cells.

As used herein, the term “modulating expression” relative to an oligonucleotide refers to the ability of an antisense oligonucleotide (oligomer) to either enhance or reduce the expression of a given protein by interfering with the expression, or translation of RNA. In the case of enhanced protein expression, the antisense oligomer may block expression of a suppressor gene, e.g., a tumor suppressor gene. In the case of reduced protein expression, the antisense oligomer may directly block expression of a given gene, or contribute to the accelerated breakdown of the RNA transcribed from that gene.

As used herein, the terms “tumor” and “cancer” refer to a cell that exhibits a loss of growth control and forms unusually large clones of cells. Tumor or cancer cells generally have lost contact inhibition and may be invasive and/or have the ability to metastasize.

As used herein, “effective amount” relative to an antisense oligomer refers to the amount of antisense oligomer administered to a mammalian subject, either as a single dose or as part of a series of doses and which is effective to inhibit expression of a selected target nucleic acid sequence.

As used herein “treatment” of an individual or a cell is any type of intervention used in an attempt to alter the natural course of the individual or cell. Treatment includes, but is not limited to, administration of e.g., a pharmaceutical composition, and may be performed either prophylactically, or subsequent to the initiation of a pathologic event or contact with an etiologic agent.

As used herein, “chemotherapeutic agent” refers to any of a number of agents with established or potential use in cancer therapy such as antimetabolites, agents that cause oxidative stress, alkylating agents, natural products, enzymes, therapeutic proteins (e.g. TRAIL) and other miscellaneous agents.

II. Cancer Therapies and Resistance to Treatment

Chemotherapeutic agents are designed to inhibit cell replication and/or cause cell death, and one way by which cells die is referred to as apoptosis, or programmed cell death. The apoptosis pathway has been highly conserved throughout evolution, and plays a critical role in embryonic development, immune function, viral pathogenesis, cancer, autoimmune disorders, and neurodegenerative disease. For example, inappropriate apoptosis may cause or contribute to AIDS, Alzheimer's Disease, Parkinson's Disease, Amyotrophic Lateral Sclerosis (ALS), retinitis pigmentosa and other diseases of the retina, myelodysplastic syndrome (e.g. aplastic anemia), toxin-induced liver disease, and ischemic injury (e.g. myocardial infarction, stroke, and reperfusion injury). Conversely, the failure of an apoptotic response has been implicated in the development of cancer, particularly follicular lymphoma, p53-mediated carcinomas, hormone-dependent tumors, prostate cancer and in the development of drug resistant cancer cells.

A. Radiation Therapy

Radiation therapy has become a foremost choice of treatment for a majority of cancer patients. The wide use of radiation treatment stems from the ability of gamma-irradiation to induce irreversible damage in targeted cells with the preservation of normal tissue function. The major practical problem associated with radiation treatment is the failure of radiotherapy due to arising tumour radioresistance (Zhivotovsky, Joseph et al. 1999). Substantial experimental evidence suggests that ionizing radiation triggers apoptosis, the intrinsic cellular death machinery in cancer cells, and the activation of apoptosis seems to be the principal mode by which cancer cells die following exposure to ionizing radiation. Disruption of apoptosis is considered to be an important step in the initiation of the tumorigenic process because cells with damaged DNA would normally be eliminated by apoptosis. Resistant tumor cells do not undergo radiation-induced apoptosis and in these cells the activation of the apoptotic machinery is typically impaired. The upregulation of XIAP resulting in increased radiation resistance and enhanced cell survival is one possible mechanism for resistance.

B. Chemotherapeutic Agents

Current cancer therapeutic regimens suffer from a number of deficiencies the most important of which are a lack of efficacy and frequent toxic side effects. One of the major limitations to clinical use of cancer therapeutic agents is the development of resistance to the treatment. The problem of drug resistance has been observed with a number of chemotherapeutic agents, including cisplatin-type compounds used to treat solid tumors and leukemias. Such resistance is typically evidenced by recurrence of the tumor subsequent to chemotherapy. As a result, most therapeutic regimes include two or more different drugs as a method of circumventing resistance. In addition, high dose chemotherapy is typically required for effective treatment. Such high doses are associated with toxic side effects.

Prostate cancer is an example of a cancer that typically progresses to chemotherapeutic drug resistance. Even with definitive therapy, most prostate cancer patients eventually progress to androgen-independent disease associated with chemotherapeutic resistance and increased mortality. Moreover, current therapies like androgen ablation have been observed to precipitate changes in gene expression profile leading to an androgen-independent phenotype (Miyake, Pollak et al. 2000).

C. TRAIL Protein

Tumor necrosis factor (TNF)-related apoptosis-inducing ligand (TRAIL) is one of several members of the TNF gene superfamily that induce apoptosis through engagement of death receptors. TRAIL is unusual as compared to any other cytokine as it interacts with a complex system of receptors including two pro-apoptotic death receptors and three anti-apoptotic decoys. This protein has generated interest as a potential tumor-specific cancer therapeutic because, as a stable soluble trimer, it selectively induces apoptosis in many transformed cells but not in normal cells. TRAIL is currently an experimental anticancer therapeutic undergoing clinical trials.

III. X-Linked Inhibitor of Apoptosis Proteins (XIAP)

X-linked Inhibitor of apoptosis proteins (XIAPs) represent a potent group of endogenous modulators of apoptosis in mammalian cells. These include a family of intracellular anti-apopototic proteins one of which is the X-linked inhibitor of apoptosis protein (XIAP). IAPs mediate multiple biological functions that include binding and inhibiting caspases, regulating the cell-cycle progression and modulating receptor-mediated signal transduction. XIAP (alternative names:MIHA/hlLP/BIRC4) has been identified as the key and most potent caspase inhibitor. Unlike Bcl2 proteins which can block only the mitochondrial branch of apoptosis by preventing release of cytochrome c, XIAP has the ability to inhibit both mitochondrial-dependent and -independent apoptotic pathways by directly binding to and inhibiting both the initiator and effector caspases (Srivastava 2001).

XIAP mRNA is about 9 kb but only 1.5 kb is the coding region leaving about 1.5 and 6 kb for the 5′ and 3′ UTR respectively. The presence of the long 5′ UTR is rare in eukaryotic transcripts and is believed to interfere with efficient translation. Interestingly, the presence of a specific sequence termed IRES (internal ribosome entry sequence involved in cap-independent translation) in the 5′ UTR facilitates efficient translation. This is the case postulated with ubiquitous expression of XIAP mRNA in most adult and fetal tissues. The IRES containing mRNAs are translated more under stress conditions like infection, growth factor deprivation, hypoxia and radiation induced apoptosis when most other cellular proteins are inhibited. The IRES sequence in XIAP has been identified to be critical for XIAP function.

The current invention is involves, in one embodiment, the manipulation of XIAP expression using novel phosphorodiamidate morpholino antisense oligomer (PMO) to induce therapeutic apoptosis and sensitize cancer cells to a cancer-therapeutic agent, e.g., radiation, chemotherapeutic agents, or TRAIL protein. In an exemplary embodiment, the PMO is used to sensitize androgen-independent cancer cells to a chemotherapeutic agent, such as cis-platin, or the TRAIL protein. In addition, the observed cisplatin-resistance and partial sensitivity to TRAIL in DU145 cells is associated with XIAP expression. XIAP inhibition using XIAP antisense PMO agent induced apoptosis and increased sensitivity of these cells to cisplatin and TRAIL.

The present invention discloses that XIAP, a potent caspase inhibitor, plays a critical role in modulating chemosensitivity in resistant cancer cells, e.g., DU145 cells, a human androgen-unresponsive and invasive prostate cancer cell model. DU145 cells constitutively express XIAP. Abrogation of XIAP along with modulation of the PI 3-K/Akt survival pathway and activation of caspase-3 has a pronounced effect on cancer cell viability.

Also in accordance with the invention, a combination treatment strategy involving the use of XIAP antisense, e.g., PMO antisense, in combination with cisplatin or TRAIL was shown to cause a significant decrease in cisplatin resistance at earlier time points and enhanced TRAIL sensitivity compared to cisplatin or TRAIL alone. These results demonstrate that the XIAP antisense antisense, e.g., PMO can enhance the effect of cisplatin and TRAIL through the combined effect of decreasing XIAP and Akt levels coupled with caspase-3 activation.

In summary, XIAP antisense, e.g., PMO XIAP antisense, downregulates XIAP in resistant cancer cells and this effect potentiates the efficacy of cytotoxic agents in a schedule-dependent manner. This novel PMO-based antisense strategy provides a non-toxic therapeutic approach to cancer treatment.

IV. Antisense Oligonucleotides for Use in Practicing the Invention

A. Preferred Antisense Oligonucleotides

Antisense oligomers for use in practicing the invention preferably have the properties: (1) a backbone that is substantially uncharged, (2) the ability to hybridize with the complementary sequence of a target RNA with high affinity, that is a Tm substantially greater than 37° C., preferably at least 45° C., and typically greater than 50° C., e.g., 60° C.-80° C. or higher, (3) a subunit length of at least 8 bases, generally about 8-40 bases, preferably 12-25 bases, (4) nuclease resistance (Hudziak, Barofsky et al. 1996). In addition, the antisense compounds have the capability for active or facilitated transport in target cells, e.g., cancer cells, as evidenced by (i) competitive binding with a phosphorothioate antisense oligomer, and/or (ii) the ability to transport a detectable reporter into target cells.

Candidate antisense oligomers may be evaluated, according to well known methods, for acute and chronic cellular toxicity, such as the effect on protein and DNA synthesis as measured via incorporation of 3H-leucine and 3H-thymidine, respectively. In addition, various control oligonucleotides, e.g., control oligonucleotides such as sense, nonsense or scrambled antisense sequences, or sequences containing mismatched bases, in order to confirm the specificity of binding of candidate antisense oligomers. The outcome of such tests is important in discerning specific effects of antisense inhibition of gene expression from indiscriminate suppression. Accordingly, sequences may be modified as needed to limit non-specific binding of antisense oligomers to non-target nucleic acid sequences.

Heteroduplex formation. The effectiveness of a given antisense oligomer molecule in forming a heteroduplex with the target mRNA may be determined by screening methods known in the art. For example, the oligomer is incubated in a cell culture containing an mRNA preferentially expressed in activated lymphocytes, and the effect on the target mRNA is evaluated by monitoring the presence or absence of (1) heteroduplex formation with the target sequence and non-target sequences using procedures known to those of skill in the art, (2) the amount of the target mRNA expressed by activated lymphocytes, as determined by standard techniques such as RT-PCR or Northern blot, (3) the amount of protein transcribed from the target mRNA, as determined by standard techniques such as ELISA or Western blotting. (See, for example, Pari, Field et al. 1995; Anderson, Fox et al. 1996).

Uptake into cells. A second test measures cell transport, by examining the ability of the test compound to transport a labeled reporter, e.g., a fluorescence reporter, into cells. The cells are incubated in the presence of labeled test compound, added at a final concentration between about 10-300 nM. After incubation for 30-120 minutes, the cells are examined, e.g., by microscopy or FACS analysis, for intracellular label. The presence of significant intracellular label is evidence that the test compound is transported by facilitated or active transport.

RNAse resistance. Two general mechanisms have been proposed to account for inhibition of expression by antisense oligonucleotides (Agrawal, Mayrand et al. 1990; Bonham, Brown et al. 1995; Boudvillain, Guerin et al. 1997). In the first, a heteroduplex formed between the oligonucleotide and the viral RNA acts as a substrate for RNaseH, leading to cleavage of the viral RNA. Oligonucleotides belonging, or proposed to belong, to this class include phosphorothioates, phosphotriesters, and phosphodiesters (unmodified “natural” oligonucleotides). Such compounds expose the viral RNA in an oligomer: RNA duplex structure to hydrolysis by RNaseH, and therefore loss of function.

A second class of oligonucleotide analogs, termed “steric blockers” or, alternatively, “RNaseH inactive” or “RNaseH resistant” , have not been observed to act as a substrate for RNaseH, and are believed to act by sterically blocking target RNA nucleocytoplasmic transport, splicing, translation, or replication. This class includes methylphosphonates (Toulme, Tinevez et al. 1996), morpholino oligonucleotides, peptide nucleic acids (PNA's), certain 2′-O-allyl or 2′-O-alkyl modified oligonucleotides (Bonham, Brown et al. 1995), and N3′→P5′ phosphoramidates (Ding, Grayaznov et al. 1996; Gee, Robbins et al. 1998).

A test oligomer can be assayed for its RNaseH resistance by forming an RNA: oligomer duplex with the test compound, then incubating the duplex with RNaseH under a standard assay conditions, as described (Stein, Foster et al. 1997). After exposure to RNaseH, the presence or absence of intact duplex can be monitored by gel electrophoresis or mass spectrometry.

In vivo uptake. In accordance with another aspect of the invention, there is provided a simple, rapid test for confirming that a given antisense oligomer type provides the required characteristics noted above, namely, high Tm, ability to be actively taken up by the host cells, and substantial resistance to RNaseH. This method is based on the discovery that a properly designed antisense compound will form a stable heteroduplex with the complementary portion of the viral RNA target when administered to a mammalian subject, and the heteroduplex subsequently appears in the urine (or other body fluid). Details of this method are also given in co-owned U.S. Pat. No. 6,365,351 for “Non-Invasive Method for Detecting Target RNA,” the disclosure of which is incorporated herein by reference.

Briefly, a test oligomer containing a backbone to be evaluated, having a base sequence targeted against a known RNA, is injected into a mammalian subject. The antisense oligomer may be directed against any intracellular RNA, including RNA encoded by a host gene. Several hours (typically 8-72) after administration, the urine is assayed for the presence of the antisense-RNA heteroduplex. If heteroduplex is detected, the backbone is suitable for use in the antisense oligomers of the present invention.

The test oligomer may be labeled, e.g. by a fluorescent or a radioactive tag, to facilitate subsequent analyses, if it is appropriate for the mammalian subject. The assay can be in any suitable-solid-phase or fluid format. Generally, a solid-phase assay involves first binding the heteroduplex analyte to a solid-phase support, e.g., particles or a polymer or test-strip substrate, and detecting the presence/amount of heteroduplex bound. In a fluid-phase assay, the analyte sample is typically pretreated to remove interfering sample components. If the oligomer is labeled, the presence of the heteroduplex is confirmed by detecting the label tags. For non-labeled compounds, the heteroduplex may be detected by immunoassay if in solid phase format or by mass spectroscopy or other known methods if in solution or suspension format.

B. Structural Features The ability to be taken up selectively by activated immune cells requires, in part, that the oligomer backbone be substantially uncharged. The ability of the oligomer to form a stable duplex with the target RNA will depend on the oligomer backbone, the length and degree of complementarity of the antisense oligomer with respect to the target, the ratio of G:C to A:T base matches, and the positions of any mismatched bases. The ability of the antisense oligomer to resist cellular nucleases promotes survival and ultimate delivery of the agent to the cell cytoplasm.

Morpholino oligonucleotides, particularly phosphoramidate- or phosphorodiamidate-linked morpholino oligonucleotides have been shown to have high binding affinities for complementary or near-complementary nucleic acids. Morpholino oligomers also exhibit little or no non-specific antisense activity, afford good water solubility, are resistant to nucleases, and are designed to have low production costs (Summerton and Weller 1997).

Morpholino oligonucleotides (including antisense oligomers) are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

In one preferred approach, antisense oligomers for use in practicing the invention are composed of morpholino subunits of the form shown in the above cited patents, where (i) the morpholino groups are linked together by uncharged linkages, one to three atoms long, joining the morpholino nitrogen of one subunit to the 5′ exocyclic carbon of an adjacent subunit, and (ii) the base attached to the morpholino group is a purine or pyrimidine base-pairing moiety effective to bind, by base-specific hydrogen bonding, to a base in a polynucleotide. The purine or pyrimidine base-pairing moiety is typically adenine, cytosine, guanine, uracil or thymine. Preparation of such oligomers is described in detail in U.S. Pat. No. 5,185,444 (Summerton et al., 1993), which is hereby incorporated by reference in its entirety. As shown in this reference, several types of nonionic linkages may be used to construct a morpholino backbone.

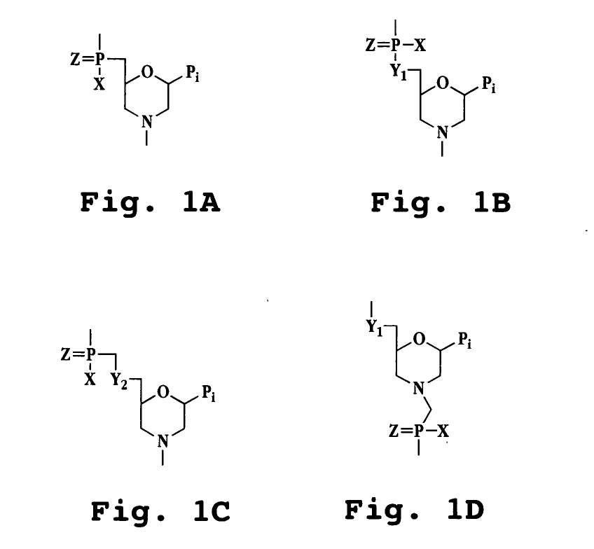

Exemplary subunit structures for antisense oligonucleotides of the invention include the morpholino subunit types shown in FIGS. 1A-D, each linked by an uncharged, phosphorous-containing subunit linkage, as shown in FIGS. 2A-2D, respectively. In these figures, the X moiety pendant from the phosphorous may be any of the following: fluorine; an alkyl or substituted alkyl; an alkoxy or substituted alkoxy; a thioalkoxy or substituted thioalkoxy; or, an unsubstituted, monosubstituted, or disubstituted nitrogen, including cyclic structures. Alkyl, alkoxy and thioalkoxy preferably include 1-6 carbon atoms, and more preferably 1-4 carbon atoms. Monosubstituted or disubstituted nitrogen preferably refers to lower alkyl substitution, and the cyclic structures are preferably 5- to 7-membered nitrogen heterocycles optionally containing 1-2 additional heteroatoms selected from oxygen, nitrogen, and sulfur. Z is sulfur or oxygen, and is preferably oxygen.

FIG. 1A shows a phosphorous-containing linkage which forms the five atom repeating-unit backbone shown in FIG. 2A, where the morpholino rings are linked by a 1-atom phosphoamide linkage. Subunit B in FIG. 1B is designed for 6-atom repeating-unit backbones, as shown in FIG. 2B. In FIG. 1B, the atom Y linking the 5′ morpholino carbon to the phosphorous group may be sulfur, nitrogen, carbon or, preferably, oxygen. The X moiety pendant from the phosphorous may be any of the following: fluorine; an alkyl or substituted alkyl; an alkoxy or substituted alkoxy; a thioalkoxy or substituted thioalkoxy; or, an unsubstituted, monosubstituted, or disubstituted nitrogen, including cyclic structures. Z is sulfur or oxygen, and is preferably oxygen. Particularly preferred morpholino oligonucleotides include those composed of morpholino subunit structures of the form shown in FIG. 2B, where X is an amine or alkyl amine of the form X═NR₂, where R is independently H or CH₃, that is where X═NH₂, X═NHCH₃ or X═N(CH₃)₂, Y═O, and Z=O.

Subunits C-D in FIGS. 1C-D are designed for 7-atom unit-length backbones as shown for structures in FIGS. 2C and D. In Structure C, the X moiety is as in Structure B, and the moiety Y may be methylene, sulfur, or preferably oxygen. In Structure D, the X and Y moieties are as in Structure B. In all subunits depicted in FIGS. 1 and 2, each Pi and Pj is a purine or pyrimidine base-pairing moiety effective to bind, by base-specific hydrogen bonding, to a base in a polynucleotide, and is preferably selected from adenine, cytosine, guanine and uracil.

As noted above, the substantially uncharged oligomer may advantageously include a limited number of charged linkages, e.g. up to about 1 per every 5 uncharged linkages. In the case of the morpholino oligomers, such a charged linkage may be a linkage as represented by any of FIGS. 2A-D, preferably FIG. 2B, where X is oxide (—O—) or sulfide (—S—).

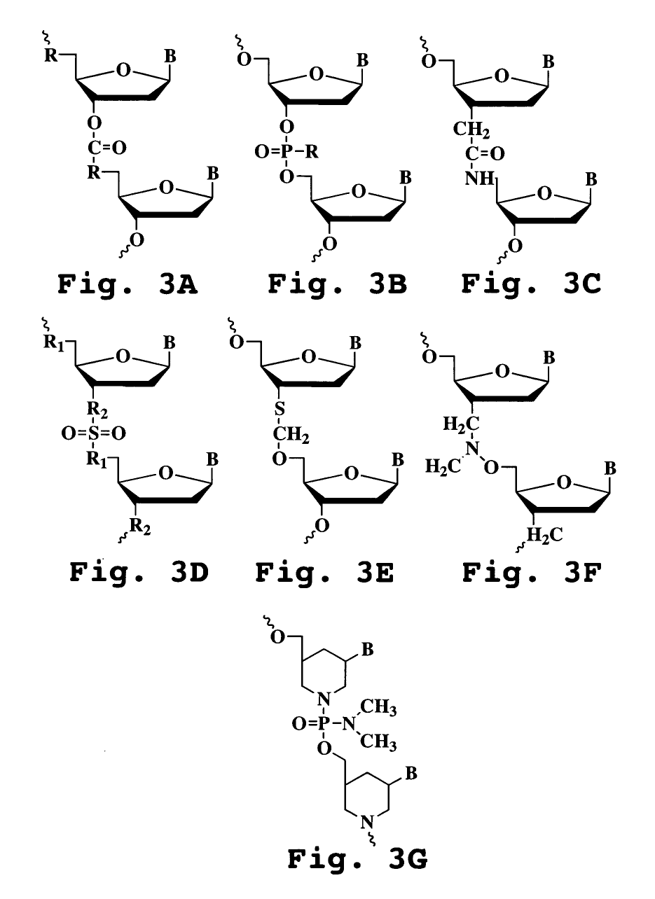

More generally, the morpholino oligomers with uncharged backbones are shown in FIGS. 3A-3G. Especially preferred is a substantially uncharged morpholino oligomer such as illustrated by the phosphorodiamidate morpholino oligomer (PMO) shown in FIG. 3G. It will be appreciated that a substantially uncharged backbone may include one or more, e.g., up to 10-20% of charged intersubunit linkages, typically negatively charged phosphorous linkages.

In addition to a base sequence complementary to a region of a selected nucleic acid target sequence, preferred antisense oligonucleotides exhibit highly specific binding to the complementary target sequence and efficacy in blocking expression of the target nucleic acid in cell and cell-free systems.

C. Preferred Antisense Targets

In practicing the invention, mRNA transcribed from the relevant region of a gene of interest is generally targeted by antisense oligonucleotides; however, single-stranded RNA, double-stranded RNA, single-stranded DNA or double-stranded DNA may be targeted. For example, double-stranded DNA may be targeted using a non-ionic probe designed for sequence-specific binding to major-groove sites in duplex DNA. Exemplary probes are described in U.S. Pat. No. 5,166,315 (Summerton and Weller, 1992), which is hereby incorporated by reference. Such probes are generally referred to herein as antisense oligomers, referring to their ability to block expression of target nucleic acids.

In the methods of the invention, the antisense oligomer is designed to hybridize to a region of the XIAP nucleic acid sequence, under physiological conditions with a Tm substantially greater than 37° C., e.g., at least 45° C. and preferably 60° C. to 80° C. The oligomer is designed to have high-binding affinity to the nucleic acid and may be 100% complementary to the XIAP target sequence or may include mismatches, e.g., to accommodate allelic variants, as long as the heteroduplex formed between the oligomer and XIAP target sequence is sufficiently stable to withstand the action of cellular nucleases and other modes of degradation during its transit from cell to body fluid. Mismatches, if present, are less destabilizing toward the end regions of the hybrid duplex than in the middle. The number of mismatches allowed will depend on the length of the oligomer, the percentage of G:C base pair in the duplex and the position of the mismatch(es) in the duplex, according to well understood principles of duplex stability.

Although such an antisense oligomer is not necessarily 100% complementary to the XIAP target sequence, it is effective to stably and specifically bind to the target sequence such that expression of XIAP is modulated. The appropriate length of the oligomer to allow stable, effective binding combined with good specificity is about 8-40 nucleotide base units, and preferably about 12-25 nucleotides. Oligomer bases that allow degenerate base pairing with target bases are also contemplated, assuming base-pair specificity with the target is maintained.

In one preferred approach, the target for modulation of gene expression using the antisense methods of the present invention comprises a sequence spanning the mRNA translational start codon for XIAP. In an alternative preferred approach, a splice acceptor or donor region of preprocessed XIAP RNA is targeted. In yet another preferred approach, the IRES region of XIAP mRNA is targeted. It will be understood that other regions of XIAP mRNA may be targeted, including one or more of, an initiator or promoter site, an intron or exon junction site, a 3′-untranslated region, and a 5′-untranslated region. It will be further understood that both spliced and unspliced RNA may serve as the template for design of antisense oligomers for use in the methods of the invention. (See, e.g., (Hudziak, Summerton et al. 2000), expressly incorporated by reference herein.)

Exemplary target sequences and antisense oligomer sequences to XIAP (targeting sequences) are provided in Tables 1 and 2, below.

TABLE 1
Exemplary XIAP Target Sequences

| | | | |
|---|---|---|---|
| | SEQ ID | GenBank | |
| Target Sequences | NO. | Acc. No. | Location |

| | | | |
|---|---|---|---|
| gagaagatgacttttaacag | 1 | AL121601 | 13779-13798 |
| cctattttcaagagaagatg | 2 | AL121601 | 13768-13787 |
| cttttaacagttttgaagg | 3 | AL121601 | 13789-13807 |
| gaaaaggtggacaagtcc | 4 | AL121601 | 13752-13769 |
| gctggattttatgctttag | 5 | AL121601 | 14643-14661 |
| gtgaaggtgataaagtaaagtgc | 6 | AL121601 | 16741-16763 |

TABLE 2
Exemplary XIAP Targeting Sequences

| | | | | |
|---|---|---|---|---|
| | | | Gen- | SEQ. |
| | Targeting sequences | Target | Bank | ID |
| Name | (5′ to 3′) | Ncts. | Acc. # | NO. |

| | | | | |
|---|---|---|---|---|
| AUG1 | CTGTTAAAAGTCATCTTCTC | 28-47 | U45880 | 7 |
| AUG2 | CATCTTCTCTTGAAAATAGG | 17-36 | U45880 | 8 |
| AUG3 | CCTTCAAAACTGTTAAAAG | 38-56 | U45880 | 9 |
| IRES | GGACTTGTCCACCTTTTC | 1-18 | U45880 | 10 |
| SD1 | CTAAAGCATAAAATCCAGC | 892-910 | U45880 | 11 |
| SA2 | GCACTTTACTTTATCACCTTCAC | 911-933 | U45880 | 12 |
| Scram- | CTTGATAGAATCTACTCTCT | NA | NA | 13 |
| ble | | | | |

In exemplary embodiments of the invention, the antisense oligomer is a PMO containing at least 6 contiguous bases of one of the sequences presented as SEQ ID NOS:7-12. Exemplary antisense sequences include those identified by SEQ ID NOS: 7-12.

V. Treatment of Cancer Using the Methods of the Invention

The invention provides methods for treatment of cancer with an antisense oligonucleotide directed against a nucleic acid sequence encoding XIAP, together with a traditional cancer treatment, i.e., chemotherapy and/or radiation therapy.

The invention is based on the discovery that a stable, substantially uncharged antisense oligonucleotide, characterized by high Tm capable of active or facilitated transport into cells, and capable of binding with high affinity to a complementary or near-complementary XIAP nucleic acid sequence, can be administered to a cancer patient, inhibit expression of XIAP by a cell, and when administered in combination with a traditional chemotherapeutic agents or newly emerging anticancer therapeutic drugs results in modulation of tumor growth.

A. Treatment of Cancer

In vivo administration of a XIAP antisense oligomer to a subject together with a traditional cancer treatment, using the methods described herein can result in an improved therapeutic outcome for the patient, dependent upon a number of factors including (1) the duration, dose and frequency of XIAP antisense oligomer administration, (2) the duration, dose, frequency and compound used for chemotherapy, (3) the duration and timing of XIAP antisense oligomer administration relative to administration of the chemotherapeutic agent, and (4) the general condition of the subject.

In general, an improved therapeutic outcome relative to a cancer patient refers to a slowing or diminution of the growth of cancer cells or a solid tumor, or a reduction in the total number of cancer cells or total tumor burden, or a favorable therapeutic outcome at a reduced dose of anti-cancer agent.

In preferred applications of the method, the subject is a human subject. The subject may also be a cancer patient, in particular a patient diagnosed as having a form of leukemia, lymphoma, neuroblastoma, breast cancer, colon cancer, lung cancer, or any type of cancer where the patient is being treated or has been treated with chemotherapy or radiation therapy. The method is also applicable to treatment of acute or chronic myelogenous leukemia, cholangiocarcinoma, melanoma, multiple myeloma, osteosarcoma, gastric sarcoma, glioma, bladder, cervical, colorectal, ovarian, pancreatic, prostrate, and stomach cancer.

Chemotherapy and/or radiation therapy alone or in combination with stem cell transplantation are standard treatment regimens for a number of malignancies, including acute lymphocytic leukemia, chronic myelogenous leukemia, neuroblastoma, lymphoma, breast cancer, prostate cancer, colon cancer, lung cancer, ovarian cancer, thymomas, germ cell tumors, multiple myeloma, melanoma, testicular cancer, lung cancer, and brain cancer.

Many cancer treatment regimens result in immunosuppression of the patient, leaving the patient with anemia, thrombocytopenia (low platelet count), and/or neutropenia (low neutrophil count). Following such cancer treatment, patients are often unable to defend against infection. Supportive care for immunosuppression may include protective isolation of the patient such that the patient is not exposed to infectious agents; administration of: antibiotics, e.g., antiviral agents and antifungal agents; and/or periodic blood transfusions to treat anemia, thrombocytopenia and/or neutropenia.

The surprising and unexpected results observed following administration of an oligomer antisense to XIAP in a combination regimen with a traditional chemotherapeutic agent suggest that XIAP may be important in maintaining the transformed phenotype and in chemoresistance in cancer cells.

A combination treatment strategy involving the use of XIAP antisense PMO in combination with cisplatin or TRAIL caused a significant decrease in cisplatin resistance at earlier time points and enhanced TRAIL sensitivity compared to cisplatin or TRAIL alone. These results provide evidence that the XIAP antisense PMO can enhance the effect of cisplatin and TRAIL through the combined effect of decreasing XIAP and Akt levels coupled with caspase-3 activation. Of particular interest are treatment regimens that combine administration of cisplatin or TRAIL and administration of an oligomer antisense to XIAP. In such treatment regimens, the chemotherapeutic agent may be administered prior to, at the same time or following administration of the antisense oligomer.

B. Chemotherapeutic Agents

Chemotherapeutic agents for use in practicing the invention include any of a number of agents with established use in cancer therapy. Exemplary chemotherapeutic agents for use in the invention are antimetabolities, compounds which cause oxidative stress, and topoisomerase inhibitors. Without being bound to any one particular theory, it is believed that chemotherapeutic agents are more toxic to less differentiated cells and as such, a population of more highly differentiated cancer cells that are refractory to the chemotherapeutic agent remain after chemotherapy treatment. Such cells may be more differentiated and accordingly, more susceptible to inhibition or cell death by a XIAP antisense oligomer.

Exemplary anticancer drugs include, but are not limited to: (1) antimetabolites such as folic acid analogs and methotrexate, (MTX); pyrimidine analogs such as 5-fluorouracil, (5-FU), fluorodeoxyuridine, cytosine arabinoside and cytarabine; purine analogs such as 6-mercaptopurine, (6-MP), 6-thioguanine, (6-TG) and 2-deoxycyoformycin (Pentostatin); (2) alkylating agents such as nitrogen mustards, mechlorethamine, cyclophosphamide (CytoxanR), Ifosfamide, melphalan, and chlorambucil; (3) natural products including, but not limited to vinca alkaloids, vincristine (OncovinR), vinblastine (VelbanR), vinorelbine (NavelbineR), epipodophylotoxins, etoposide (VePesidR, VP-16) and taxol (PaclitaxeiR); (4) compounds characterized as anti-tumor antibiotics which include, but are not limited to anthracyclines, doxorubicin hydrochloride, (adriamycinR), daunorubicin, idarubicin, mitoxantrone, bleomycin, (blenoxaneR), dactinomycin (actinomycin D), mitomycin C, plycamycin and (mithramycin); and (5) miscellaneous agents including, but not limited to cisplatin, carboplatin, asparaginase, hydroxyurea, mitotane (o,p′-DDD; Lysodren), tamoxifen, and prednisone.

Cisplatin (also called cis-platinum, platinol; cis-diamminedichloroplatinum; and cDDP) is representative of a broad class of water-soluble, platinum coordination compounds frequently employed in the therapy of testicular cancer, ovarian tumors, and a variety of other cancers. (See, e.g., Blumenreich, Woodcock et al. 1985; Forastiere, Leong et al. 2001). Methods of employing cisplatin clinically are well known in the art. For example, cisplatin has been administered in a single day over a six hour period, once per month, by slow intravenous infusion. For localized lesions, cisplatin can be administered by local injection. Intraperitoneal infusion can also be employed. Cisplatin can be administered in doses as low as 10 mg/m² per treatment if part of a multi-drug regimen, or if the patient has an adverse reaction to higher dosing. In general, a clinical dose is from about 30 to about 120 or 150 mg/m² per treatment.

Typically, platinum-containing chemotherapeutic agents are administered parenterally, for example by slow intravenous infusion, or by local injection, as discussed above. The effects of intralesional (intratumoral) and IP administration of cisplatin is described in (Nagase, Nomura et al. 1987; Theon, Pascoe et al. 1993).

Although cisplatin is widely used, side effects reported following administration of cisplatin are common and include thinned or brittle hair, loss of appetite and/or weight, diarrhea, nausea and vomiting, and numbness or tingling in the fingertips and toes. In general, the effects of cisplatin are non-specific and administration of cisplatin results in damage to all rapidly growing tissues. See, e.g., Gandara, Perez et al. 1991; Byhardt 1995; Jones and Chesney 1995; Peters, van der Wilt et al. 2000).

Further, cisplatin is effective against a narrow range of tumors and the development of resistance has been reported (Onoda, Nelson et al. 1988). Taxol (Paclitaxel) is a complex diterpenoid originally isolated in small yields from the bark of various species of yew (Taxaceae). Taxol can now also be prepared by chemical synthesis. (See, e.g., Nicolaou, Yang et al. 1994). Taxol constitutes one of the most potent drugs in cancer chemotherapy and has been approved by FDA for treatment of ovarian and breast cancer and has exhibited potential utility in the treatment of lung, skin, and head/neck cancers.

The clinical utility of taxol and related drugs has been limited by cost, limited bioavailability (due to of low aqueous solubility), and the development of multiresistant cells. Solubilizers, such as Cremophor (polyethoxylated castor oil) and alcohol have been demonstrated to improve the solubility and microencapsulated forms have been described. (See, e.g., WO 93/18751) In general, side effects reported for taxol (paclitaxel), include a reduction in white and red blood cell counts, infection, nausea and vomiting, loss of appetite, change in taste, hair loss, joint and muscle pain, numbness in the extremities and diarrhea.

Etoposide (etoposide (VP-16, VePesid Oral) is currently used in therapy for a variety of cancers, including testicular cancer, lung cancer, lymphoma, neuroblastoma, non-Hodgkin's lymphoma, Kaposi's Sarcoma, Wilms' Tumor, various types of leukemia, and others.

Etoposide is generally administered orally or intravenously. Side effects associated with administration of Etoposide Oral (VP-16, VePesid Oral) include nausea and vomiting, loss of appetite, diarrhea, stomach pain, fatigue and hair loss. The primary dose-limiting side effect of etoposide and related compounds is neutropenia, which is often severe, particularly among patients under treatment with additional chemotherapeutic agents or radiation.

5-FU, (Fluorouracil, Tradenames: 5-FU, Adrucil) has been used for chemotherapy for a variety of cancers, including colon cancer, rectal cancer, breast cancer, stomach cancer, pancreatic cancer, ovarian cancer, cervical cancer, bladder cancer vaginal warts, and actinic keratosis (a type of precancerous skin lesion). 5-FU is typically administered by intravenous (IV) injection, IV infusion (drip), orally, or as a cream applied directly to the skin. 5-FU has been associated with widely documented side effects including hair loss, headache, weakness, achiness, sensitivity of skin to sunlight, blistering skin or acne, loss of appetite and/or weight and tingling in the hands or feet.

C. Treatment Regimens

The present invention provides methods for cancer therapy, where an oligomer antisense to XIAP and one or more chemotherapeutic agents are administered to a patient. In a preferred aspect of the methods described herein, the XIAP antisense oligomer is administered to the patient prior to or following, but not at the same time as administration of the one or more chemotherapeutic agents.

In one preferred embodiment, cisplatin is administered to the patient prior to, or following, but not at the same time as, administration of the XIAP antisense oligomer.

In one exemplary embodiment, cisplatin is administered daily for 1 to 5 and preferably 3 consecutive days, followed by one or more days where no anti-cancer treatment is administered, then an oligomer antisense to XIAP is administered daily for 2 to 7 and preferably 5 consecutive days, with the cycle of chemotherapy and antisense oligomer administration repeated at least 2 times. In another exemplary embodiment, cisplatin, is administered daily for 1 to 5 and preferably 3 consecutive days, followed by administration of an oligomer antisense to XIAP daily for 2 to 7 and preferably 5 consecutive days, with the cycle of chemotherapy and antisense oligomer administration repeated at least 2 times.

In another exemplary embodiment, an oligomer antisense to XIAP is administered for 2 to 7 and preferable 5 consecutive days, followed by administration of TRAIL daily for 1 to 5 and preferable 3 consecutive days, with the cycle of XIAP antisense oligomer administration and TRAIL therapy repeated at least 2 times.

In another preferred embodiment, the oligomer antisense to XIAP and chemotherapeutic agent are administered sequentially and at separate times spaced by at least one day. Preferably, the oligomer antisense to XIAP is administered daily for at least two days, followed by the administration of a chemotherapeutic agent for one or more days, with the cycle of alternating administration of the antisense oligomer to XIAP and the chemotherapeutic agent repeated at least two times. The time interval between administration of the two compounds is preferably at least three times the half-life of the last administered compound, to ensure that the last-administered compound is largely cleared from the patient before administration of the other compound. Typically, chemotherapeutic compounds are cleared with a half-life of 2-6 hours, so about 6-24 hours should be allowed for clearance. The oligomer antisense to XIAP is typically cleared with a half-life of 18-24 hours so a period of 2-3 days would be allowed for clearance.

As will be understood by those of skill in the art, the optimal treatment regimen will vary and it is within the scope of the treatment methods of the invention to evaluate the status of the disease under treatment and the general health of the patient prior to, and following one or more cycles of chemotherapy and antisense oligomer administration in order to determine if additional cycles of chemotherapy and antisense oligomer administration are indicated. Such evaluation is typically carried out by use of tests typically used to evaluate traditional cancer chemotherapy, as further described below in the section entitled “Monitoring Treatment”.

The preferred treatment regimens for use in practicing the invention generally include administration of the one or more chemotherapeutic agents prior to administration of a XIAP antisense oligomer. While the mechanism is not part of the invention, following chemotherapy a population of cancer cells that are refractory to the chemotherapy remain and such cells may be more differentiated and accordingly more susceptible to modification by a XIAP antisense oligomer that is administered following chemotherapy.

As detailed above, preferred antisense oligonucleotides for use in these methods are substantially uncharged phosphorodiamidate morpholino oligomers (PMOs), characterized by stability, high Tm, and capable of active or facilitated transport as evidenced by (i) competitive binding with a phosphorothioate antisense oligomer, and/or (ii) the ability to transport a detectable reporter into the cells.

In one preferred aspect of this embodiment, the oligomer is a PMO selected from the group consisting of the sequences presented as SEQ ID NOS:7-12.

D. Delivery of Chemotherapeutic Agents

An important aspect of the invention is effective delivery of one or more chemotherapeutic agents in a pharmaceutically acceptable carrier. In accordance with one aspect of the invention, the choice of chemotherapeutic agent(s) and corresponding route and timing of delivery take advantage of one or more of: (i) established use in treatment of the particular type of cancer under treatment; (ii) the ability of the selected chemotherapeutic agent to result in an improved therapeutic when administered in combination with an oligomer antisense to XIAP; and (iii) local delivery of the chemotherapeutic agent by a mode of administration effective to achieve sufficient localized exposure of the agent to cancer cells.

In practicing the invention, the chemotherapeutic agent is administered by a route and using a treatment regimen that has an established use in cancer chemotherapy. As set forth above, the optimal route will vary with the chemotherapeutic agent. However, preferred routes typically include slow intravenous infusion (IV drip), oral administration and local injection. The formulations are easily administered in a variety of dosage forms such as injectable solutions, drug release capsules, implants or in combination with carriers such as liposomes or microcapsules.

Recommended dosages and dosage forms for a large number of chemotherapeutic agent have been established and can be obtained form conventional sources, such as the Physicians Desk Reference, published by Medical Economics Company, Inc., Oradell, N.J. If necessary, these parameters can be determined for each system by well-established procedures and analysis, e.g., in clinical trials.

For example, when orally administered, the active compounds may be combined with an inert diluent or in an edible carrier, or enclosed in hard or soft shell gelatin capsules, compressed into tablets, incorporated directly into food, incorporated with excipients and used in the form of ingestible tablets, buccal tables, troches, capsules, elixirs, suspensions, syrups, wafers, and the like. The appropriate amount of active compound is specific to the particular chemotherapeutic agent and is generally known in the art. The amount of active compound in such therapeutically useful compositions will be such that a suitable dosage is obtained.

Parenteral administration, may be accomplished using a suitable buffered aqueous solution and the liquid diluent which has been prepared in isotonic form using saline or glucose. Such aqueous solutions are appropriate for intravenous, intramuscular, subcutaneous and intraperitoneal administration. (See, for example, “Remington's Pharmaceutical Sciences”, 15th Edition, pages 1035-1038 and 1570-1580). Sterile injectable solutions are prepared by incorporating the chemotherapeutic agent in the required amount of an appropriate solvent with various other ingredients included, followed by filter sterilization. Sterile powders for use in sterile injectable solutions may be prepared by vacuum drying or freeze drying techniques or other means to result in a powder of the active chemotherapeutic agent plus additional desired ingredients prepared from a previously sterile solution.

It will be understood that the invention contemplates treatment regimens that include the administration of one or more chemotherapeutic agents and administration of an oligomer antisense to XIAP for chemotherapy of cancer. Such a treatment regimen may be administered prior to, contemporaneously with, or subsequent to additional cancer treatment, such as radiation therapy, further chemotherapy and/or immunotherapy.

The present invention provides the advantage that the dose of the one or more chemotherapeutic agents may be decreased when administered in a treatment regimen that also includes XIAP antisense oligomer administration relative to treatment regimens that do not include XIAP antisense oligomer administration. Such combination treatments are advantageous in patients that are young or old or whose cancer is recalcitrant to treatment regimens that do not include XIAP antisense oligomer administration.

E. Delivery of Antisense Oligomers to the Patient

Effective delivery of an antisense oligomer to the target XIAP nucleic acid sequence is an important aspect of the methods of the invention. In accordance with one aspect of the invention, the modes of administration discussed below exploit one of more of the key features: (i) use of an antisense compound that has a high rate of cell uptake, (ii) the ability of the antisense compound to interfere with XIAP mRNA processing and mRNA translation, and (iii) delivery of the antisense oligomer by a mode of administration effective to achieve high localized concentration of the compound to cancer cells.

In accordance with the invention, effective delivery of an oligomer antisense to XIAP may include, but is not limited to, various systemic routes, including oral and parenteral routes, e.g., intravenous, subcutaneous, intraperitoneal, and intramuscular; as well as inhalation and transdermal delivery. It is appreciated that any methods effective to deliver a XIAP antisense oligomer to into the bloodstream of a subject are also contemplated.

Transdermal delivery of antisense oligomers may be accomplished by use of a pharmaceutically acceptable carrier adapted for e.g., topical administration. One example of morpholino oligomer delivery is described in PCT patent application WO 97/40854, incorporated herein by reference.

The amount of the XIAP antisense oligonucleotide and the chemotherapeutic agent administered is such that the combination of the two types of agents is therapeutically effective. Dosages will vary in accordance with such factors as the age, health, sex, size and weight of the patient, the route of administration, the toxicity of the drugs, and the relative susceptibilities of the cancer to the oligonucleotide and chemotherapeutic agent.

Typically, one or more doses of antisense oligomer are administered, generally at regular intervals for a period of about one to two weeks. Preferred doses for oral administration are from about 1 mg oligomer/patient to about 25 mg oligomer/patient (based on an adult weight of 70 kg). In some cases, doses of greater than 25 mg oligomer/patient may be necessary. For IV administration, the preferred doses are from about 0.5 mg oligomer/patient to about 10 mg oligomer/patient (based on an adult weight of 70 kg). The antisense compound is generally administered in an amount sufficient to result in a peak blood concentration of at least 200-400 nM antisense oligomer. Greater or lesser amounts of oligonucleotide may be administered as required and maintenance doses may be lower.

In general, the method comprises administering to a subject, in a suitable pharmaceutical carrier, an amount of the antisense agent effective to inhibit expression of the XIAP nucleic acid target sequence.

It follows that the antisense oligonucleotide composition may be administered in any convenient vehicle, which is physiologically acceptable. Such an oligonucleotide composition may include any of a variety of standard physiologically acceptable carriers employed by those of ordinary skill in the art. Examples of such pharmaceutical carriers include, but are not limited to, saline, phosphate buffered saline (PBS), water, aqueous ethanol, emulsions such as oil/water emulsions, triglyceride emulsions, wetting agents, tablets and capsules. It will be understood that the choice of suitable physiologically acceptable carrier will vary dependent upon the chosen mode of administration. In some instances liposomes may be employed to facilitate uptake of the antisense oligonucleotide into cells. (See, e.g. Lappalainen, Urtti et al. 1994; Williams, Camilleri et al. 1996; Lou, Garrett et al. 2001). Hydrogels may also be used as vehicles for antisense oligomer administration, for example, as described in WO 93/01286. Alternatively, the oligonucleotides may be administered in microspheres or microparticles. Sustained release compositions are also contemplated within the scope of this application. These may include semipermeable polymeric matrices in the form of shaped articles such as films or microcapsules.

It will be understood that the effective in vivo dose of a XIAP antisense oligonucleotide for use in the methods of the invention will vary according to the frequency and route of administration as well as the condition of the subject under treatment. Accordingly, such in vivo therapy will generally require monitoring by tests appropriate to the condition being treated and a corresponding adjustment in the dose or treatment regimen in order to achieve an optimal therapeutic outcome.

In one preferred embodiment, the oligomer is a phosphorodiamidate morpholino oligomer (PMO), contained in a pharmaceutically acceptable carrier, and delivered orally. In a further aspect of this embodiment, a morpholino XIAP antisense oligonucleotide is administered at regular intervals for a short time period, e.g., daily for two weeks or less. However, in some cases the antisense oligomer is administered intermittently over a longer period of time.

In some cases, the treatment regimen will include further intervention such as radiation therapy, immunotherapy and/or additional chemotherapy. Such treatment may occur prior to, during or subsequent to administration of the chemotherapeutic agent and XIAP antisense oligomer.

VI. Evaluating the Effect of Antisense Oligomers

A. Analysis of the Effects of Antisense Oligomer Treatment

Candidate antisense oligomers are evaluated, according to well known methods, for acute and chronic cellular toxicity, such as the effect on protein and DNA synthesis as measured via incorporation of ³H-leucine and ³H-thymidine, respectively. In addition, various control oligonucleotides, e.g., control oligonucleotides such as sense, nonsense or scrambled antisense sequences, or sequences containing mismatched bases, in order to confirm the specificity of binding of candidate antisense oligomers. The outcome of such tests are important to discern specific effects of antisense inhibition of gene expression from indiscriminate suppression. (See, e.g. Bennett and Schwartz 1995). Accordingly, sequences may be modified as needed to limit non-specific binding of antisense oligomers to non-target sequences.

The effectiveness of a given antisense oligomer molecule in forming a heteroduplex with the target RNA may be determined by screening methods known in the art. For example, the oligomer is incubated a cell culture expressing XIAP, and the effect on the target RNA is evaluated by monitoring the presence or absence of (1) heteroduplex formation with the target sequence and non-target sequences using procedures known to those of skill in the art, (2) the amount of XIAP mRNA, as determined by standard techniques such as RT-PCR or Northern blot, or (3) the amount of XIAP protein, as determined by standard techniques such as ELISA or immunoblot (e.g. Western blot).

VII. Monitoring Treatment

The efficacy of a given therapeutic regimen involving the methods described herein, may be monitored, e.g., using diagnostic techniques appropriate to the type of cancer under treatment.

The nature of an evaluation will vary dependent upon the condition being treated and the treatment regimen may be adjusted (dose, frequency, route, etc.), as indicated, based on the results of such diagnostic tests.

It will be understood that an effective in vivo treatment regimen using the antisense oligonucleotides of the invention will vary according to the frequency and route of administration, as well as the condition of the subject under treatment (i.e., prophylactic administration versus administration in response to localized or systemic infection). Accordingly, such in vivo therapy will generally require monitoring by tests appropriate to the particular type of condition, e.g., cancer, under treatment and a corresponding adjustment in the dose or treatment regimen in order to achieve an optimal therapeutic outcome.

Diagnosis and monitoring of cancer generally involves one or more of (1) biopsy, (2) ultrasound, (3) x-ray, (4) magnetic resonance imaging, (5) nucleic acid detection methods, (6) serological detection methods, i.e., conventional immunoassay and (7) other biochemical methods. Such methods may be qualitative or quantitative.

The efficacy of a given therapeutic regimen involving the methods described herein may be monitored, e.g., by general indicators of the disease condition under treatment, as further described above.

Nucleic acid probes may be designed based on XIAP or other nucleic acid sequences associated with the particular cancer under treatment. Nucleic amplification tests (e.g., PCR) may also be used in such detection methods. It will be understood that the exact nature of diagnostic tests as well as other physiological factors indicative of a disease condition will vary dependent upon the particular condition being treated and whether the treatment is prophylactic or therapeutic.

In cases where the subject has been diagnosed as having a particular type of cancer, the status of the cancer is also monitored using diagnostic techniques typically used by those of skill in the art to monitor the particular type of cancer under treatment.

The antisense oligomer treatment regimen may be adjusted (dose, frequency, route, etc.), as indicated, based on the results of immunoassays, other biochemical tests and physiological examination of the subject under treatment.

VIII. Applications/Utility of the Invention

As described herein, treatment of cancer with an XIAP antisense oligonucleotide in combination with traditional cancer treatment such as chemotherapy and/or radiation therapy find utility in slowing or eliminating the growth and/or spread of the cancer. For example, the methods of the invention can: (1) inhibit or arrest the growth of cancer cells; (2) allow for lower dose and/or shorter term administration of chemotherapeutic agents resulting in a decrease in toxic side effects; (3) allow for lower dose or shorter term administration of chemotherapeutic agents decreasing the likelihood of development of resistance to the chemotherapeutic agent; (4) provide a type of antisense oligomer (e.g., a PMO) that is substantially uncharged and does not coprecipitate with the chemotherapeutic agent; and (5) provide an alternative and efficacious treatment regimen for patient populations that cannot tolerate doses of a chemotherapeutic agent required for efficacy when administered in a treatment regimen that lacks XIAP antisense oligomer administration.

All patent and literature references cited in the present specification are hereby incorporated by reference in their entirety.

The following examples illustrate but are not intended in any way to limit the invention.

### Material and Methods

Oligomers

XIAP antisense and scrambled PMOs were synthesized and purified at AVI BioPharma, Inc (Corvallis, Oreg.) with purity greater than 95% as determined by reverse phase high-performance liquid chromatography (HPLC) and MALDI TOF mass spectroscopy. The nucleotide sequence of the XIAP antisense and scrambled oligomers are 5′-CTG TTA AAA GTC ATC TTC TC-3′ (SEQ ID NO:1) and 5′-CTT GAT AGA ATC TAC TCT CT-3′ (SEQ ID NO:5), respectively. The lyophilized PMOs are water-soluble and were dissolved in sterile distilled water for in vitro experiments.

Cell Culture

DU145 human prostate cancer cells were obtained from ATCC (Manassas, Va.). RPMI-1640 culture media was purchased from Hyclone Laboratories, Inc. Logan, Utah. Penicillin and streptomycin were obtained from Gibco, Grand Island, N.Y. The cells were routinely cultured in RPMI-1640 supplemented with 10% Fetal Bovine Serum (Hyclone Laboratories, Inc, Logan, Utah), 10 U/mL penicillin and 10 μg/mL streptomycin in a 5% CO₂ and 95% air humidified incubator at 37° C.

Cell Viability Assay

Cells were seeded at 4000 cells/well in a 96-well plate (Becton Dickinson and Company, Franklin Lakes, N.J.) and allowed to reach at least 80% confluence. After treatment with agents, culture media was aspirated and 3-(4,5-dimethylthiazol-2-yl)-2,5-diphenyltetrazolium bromide [MTT] (Sigma St Louis, Mo.) was added to each well at a concentration of 0.5 mg/mL in media from a 5 mg/mL stock. The cells were incubated at 37° C. until the MTT reaction caused the formation of granulated purple coloration. Excess reagent was aspirated and replaced with 200 μl of dimethyl sulfoxide (Sigma St Louis, Mo.) in each well. The absorbance was then read at 540 nm in a Molecular Devices plate reader.

Protein Expression

Cells were harvested and immediately lysed in solubilization buffer (20 mM Hepes, pH 7.4; 150 mM NaCl; 1% deoxycholic acid; 1% Triton X-100; 0.2% sodium dodecyl sulfate [SDS] and protease inhibitor). Protein estimation was carried out by the BCA protein assay protocol (PIERCE, Rockford, Ill.). Equal amounts of protein in cell lysates were subjected to SDS-polyacrylamide gel electrophoresis (SDS-PAGE) under reducing conditions. Before loading onto the gel, all lysates except those for XIAP immunodetection were heated at 95-100° C. for 5 minutes and immediately cooled on ice. The protein was then transferred onto Immobilon-P transfer membranes (Millipore Corporation, Bedford, Mass.) previously soaked in methanol and transfer buffer. After the transfer process was complete, the membranes were allowed to dry, resoaked in methanol and then incubated with blocking buffer (4% dry non-fat milk in 1×PBS/0.1% Tween 20) for 1 h at room temperature. The membranes were incubated with primary antibody against XIAP [1:250 dilution] (BD Transduction Laboratories, Lexington, Ky.), and Akt [1:1000] (Cell Signalling Technology, Beverly, Mass.) for 1 h at room temperature and caspase-3 [1:100 dilution] (Oncogene Research Products, San Diego, Calif.) and caspase-7 [1:1000] (DB Pharmingen, San Diego, Calif.) overnight at 4° C. Membranes were washed three times with wash buffer (1×PBS/0.1% Tween 20) and subsequently incubated with appropriate secondary antibody conjugated with horseradish peroxidase (1:2000 dilution) for 1 h at room temperature. The membranes were washed three times and immunoreactive bands were visualized using epichemiluminescence (ECL) detection system (Amersham Pharmacia Biotech, UK Limited, England) and signals were developed after exposure to X-ray film (X-Omat films, Eastman Kodak Company, Rochester, N.Y.). β-actin immunodetection was conducted to serve as loading control. This was done by stripping the same membrane in stripping buffer (100 mM 2-mercaptoethanol, 2% SDS, 62.5 mM Tris-HCl, pH 6.7) at 50° C. for 30 minutes and then followed by washing and blocking procedure as indicated above (Devi, Oldenkamp et al. 2002).

Treatment of Cells with Cisplatin and TRAIL

Cells were treated for 24 h in serum-containing media in a 96-well plate (Becton Dickinson and Company, Franklin Lakes, N.J.) with cis-platinum (II) Diamine Dichloride (cisplatin)[0-250 μM (Sigma, St Louis, Mo.)], Taxol [0-1000 nM (CALBIOCHEM, San Diego, Calif.)], and recombinant TNF-α-related apoptosis-inducing ligand (TRAIL) [0-500 ng/ml (BIOMOL Research Laboratories, Inc, Plymouth, Pa.)]. Cell viability was then determined by MTT assay.

Plasmid-Based Test System for Screening PMO Antisense Activity

A fusion construct was generated by subcloning 29 bases of the 5′ untranslated region, AUG translation start site and the first 16 bases of the protein coding sequence of XIAP gene followed by luciferase reporter gene into the pCiNeo expression vector (Promega, Madison, Wis.). The fusion construct was designated pCiNeoXIAP-lucΔA. This plasmid features a T7 promoter capable of generating in vitro transcribed RNA from a cloned insert for use in the cell free rabbit reticulocyte in vitro translation reactions and a CMV promoter for constitutive expression in mammalian cells.

Luciferase Assay in Cell Culture

Confluent HeLa cells were transiently transfected with pCiNeoXIAP-lucΔA plasmid construct using Lipofectamine (Invitrogen Life Technologies, Carlsbad Calif.) according to the manufacturer's directions. Cells were trypsinized after 24 h and plated in six-well plates (Becton Dickinson and Company, Franklin Lakes, N.J.) at a density of 6×10⁵ cells/well, allowed to adhere overnight and scrape loaded with vehicle or PMOs at different concentrations. Cell lysates were prepared 24 h later, normalized for protein content and luciferase activity was determined using the luminometer.

Delivery of Antisense PMO into Cells in Culture

The antisense and scrambled PMOs were delivered into the cells by scrape loading as described earlier (Devi, Oldenkamp et al. 2002). Briefly, cells were plated at a density of 5×10⁵ cells per well in a Falcon six-well plate (Becton Dickinson and Company, Franklin Lakes, N.J.) and allowed to reach at least 95% confluence. The PMOs were diluted in 2 mL culture medium to yield the desired concentration. The cells were then scraped with a sterile scraper (Sarstedt, Newton, N.C.) in the medium, mixed gently and transferred into appropriately labeled fresh six-well plates.

Scheduled Treatment with Cis-Platinum (II) Diamine Dichloride (Cisplatin), TNF-Related Apoptosis-Inducing Ligand (TRAIL) and XIAP Antisense PMO

Cells were treated with increasing concentrations of cisplatin and TRAIL. In one case cisplatin treatment was conducted for a 24-h period and this was followed by XIAP antisense or scrambled PMOs by scrape loading for 24 h. In the study involving treatment with TRAIL, cells were first treated with XIAP antisense or scrambled PMOs by scrape loading for 24 h. This was then followed by a 24-h treatment with TRAIL. TRAIL, cisplatin and vehicle controls were also sham scrape loaded. All treatments were carried out in six-well plates at 37° C. in serum-containing culture media.

Apoptosome ELISA-Based Assay

The M30-Apoptosense™ ELISA kit (PEVIVA AB, Bromma, Sweden) was used to quantify apoptosis by detecting the levels of M30 antigen that is formed during apoptosis as a result of cleavage of cytokeratin 18 by active caspase-3. DU145 cells were treated with XIAP antisense and scrambled PMOs. Total protein extracts were prepared and 25 μl of these were added to 75 μl of diluted HRP Conjugate Solution per well of Coated Microstrips. This was allowed to react on a shaker at room temperature for 4 h. Each well was then washed three times with Wash Solution and 200 μl of TMB Substrate Solution was added to each well and incubated in darkness for 20 minutes. The reaction was stopped by adding 50 μl of Stop Solution and placed on a shaker for 5-10 seconds. An additional 5-minute period was allowed before absorbance readings were taken at 450 nm on a Molecular Devices plate reader.

### EXAMPLE 1

### Cisplatin Resistance in DU145 Cells Correlated with XIAP Expression

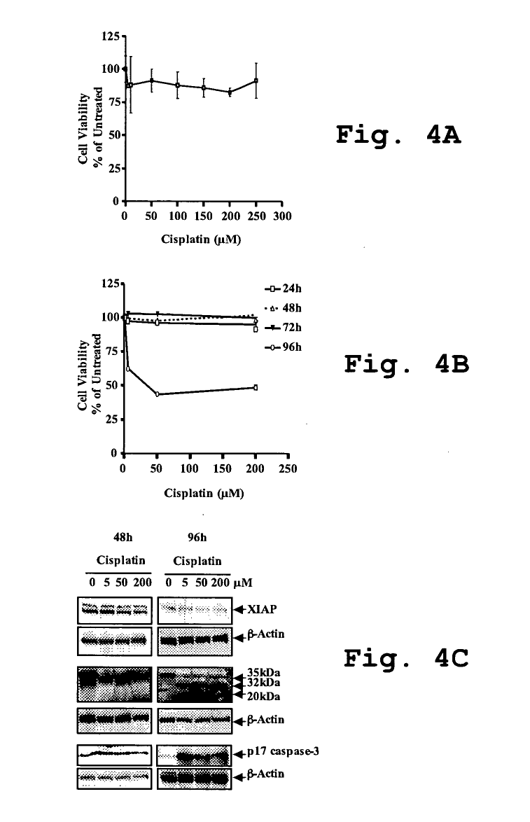

Human androgen-independent DU145 cells were treated with various concentrations of cisplatin for 24 h. The data in FIG. 4A, reveal that these cells are highly resistant to cisplatin up to 72 h even at higher concentrations (200 μM). To determine whether the effect of cisplatin on DU145 cells is time-dependent, a time course study was carried out. A dramatic 60-70% decrease in cell viability (IC₅₀=30 μM) was observed when the cells were incubated in the presence of cisplatin for 96 h (FIG. 4B). To provide a mechanistic insight into the effect of cisplatin on DU145 cell viability, we examined the expression of XIAP and caspases at various time points post-cisplatin treatment. No significant changes were observed in XIAP or active caspase-3 levels in lysates from cells treated up to 48 h with cisplatin. In contrast, a significant decrease in XIAP expression, and inactive forms of caspase-3 and -7 with a corresponding increase in active forms was observed in the lysates of the cells incubated (96 h) with cisplatin (FIG. 4C). These data suggest that cisplatin-induced cell death after 96 h of treatment may be mediated by downregulation of XIAP along with activation of caspases.

### EXAMPLE 2

### TRAIL Activates Caspase-3 but Does Not Change XIAP Levels in DU145 Cells

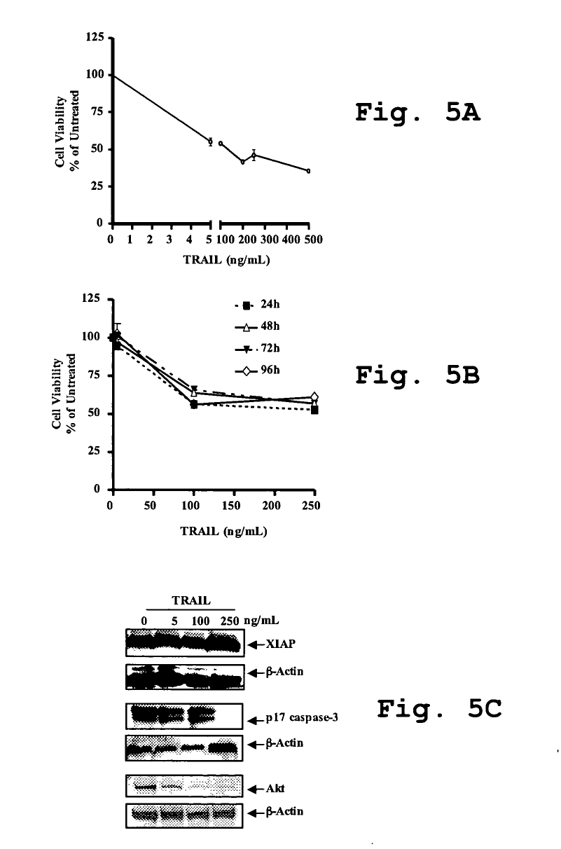

TRAIL has been shown to induce programmed cell death through death receptors-DR4 and/or DR5 followed by activation of caspases, particularly caspase-3. Treatment of DU145 cells with TRAIL caused a rapid but partial sensitivity (50% cell death) with IC₅₀=140 ng/mL. However, there was no shift in IC₅₀ or decreased cell viability with increasing concentration or duration of TRAIL treatment (FIGS. 5A and 5B). Although TRAIL significantly increased the levels of active form of caspase-3 and decreased Akt levels within 6 h, no change in XIAP expression was observed even with prolonged incubation as shown in FIG. 5C.

### EXAMPLE 3

### XIAP Antisense PMO Decreases XIAP and AKT Expression and Induces Apoptosis in DU145 Human Prostate Cancer Cells

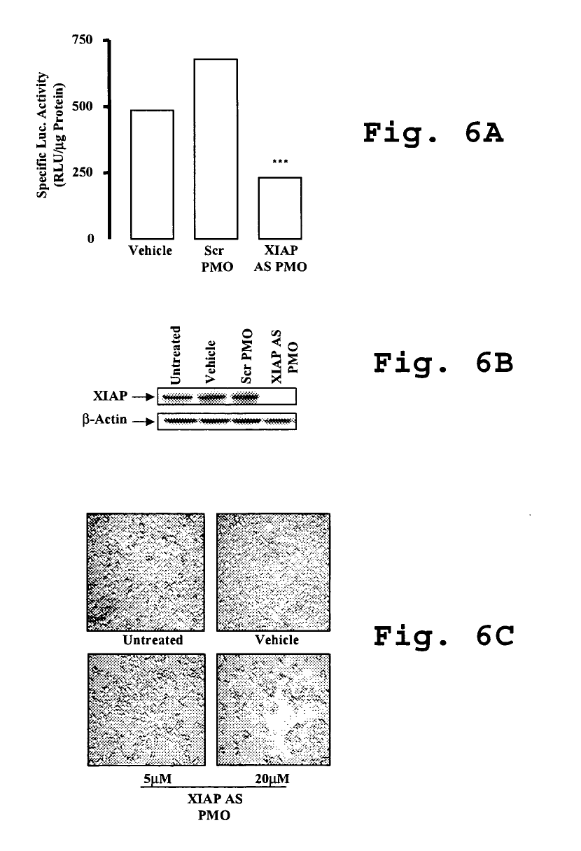

In order to examine the role of XIAP in the regulation of apoptosis, a 20 mer antisense PMO agent was generated targeting the translational start site of XIAP mRNA (SEQ ID NO:1). A plasmid-based screening system to screen antisense specificity and activity was generated by subcloning 29 bases of the 5′ untranslated region, AUG translational start-site and the first 16 bases of the protein coding sequence of XIAP gene followed by the luciferase reporter gene. Confluent HeLa cells were then transiently transfected with this plasmid construct. This experiment was conducted to confirm that antisense activity was specific to the XIAP antisense PMO sequence. The data in FIG. 6A revealed sequence-specific inhibition of luciferase activity since the antisense PMO produced a significant inhibitory effect compared to the scrambled control.

In addition, to study the effect of XIAP antisense PMO on endogenous XIAP levels, DU145 cells were treated with XIAP antisense or scrambled PMO by scrape loading with appropriate controls. Immunoblot analysis of lysates from the cells treated with XIAP antisense PMO for 24 h showed a downregulation of endogenous XIAP expression as well as a dose-dependent decrease in cell viability as shown in FIGS. 6B and 6C respectively.

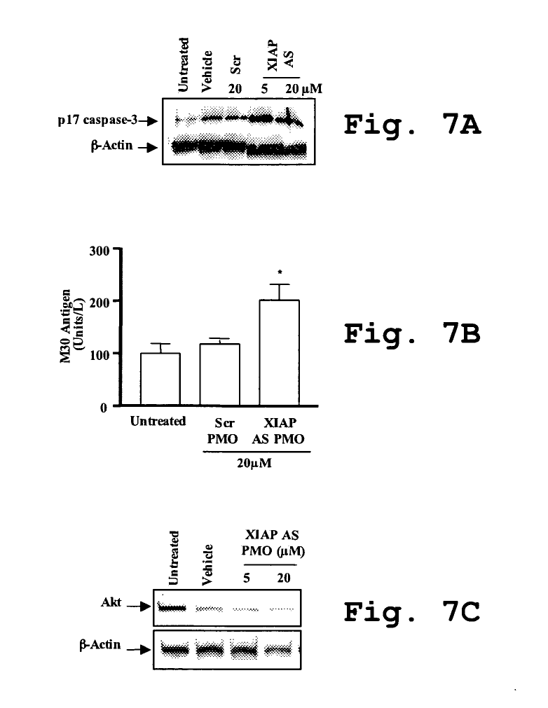

To confirm whether the observed decrease in cell viability was mediated by apoptosis, immunoblot analysis was conducted to examine the status of caspase-3. The data in FIG. 7A showed that XIAP antisense PMO induced activation of caspase-3 indicating that donwregulation of XIAP relieves the trigger block on caspase-3 activation and hence apoptosis.

Activation of capsase-3 was further confirmed by the M30 ELISA-based method that detects the presence of M30 antigen formed during apoptosis due to cleavage of cytokeratin 18, a caspase-3 substrate. The increase in M30 antigen levels observed in the XIAP antisense PMO treated cells compared to scrambled control as shown in FIG. 7B strengthens the data in FIG. 7A.

Also examined was the effect of XIAP down regulation on Akt levels. The data in FIG. 7C shows a decrease in Akt levels in the presence of XIAP antisense PMO. These data demonstrate that the observed decrease in cell viability induced by XIAP antisense PMO is mediated by a decrease in XIAP and Akt expression accompanied by activation of caspase-3.

### EXAMPLE 4

### XIAP Antisense PMO Potentiates Cisplatin and TRAIL in DU145 Human Prostate Cancer Cells

Since XIAP expression was observed to be the common link in cisplatin and TRAIL sensitivity in DU145 cells, we studied the effect of a combination treatment schedule consisting of cytotoxic agent and the XIAP antisense PMO. In one case, cells were treated with cisplatin for 24 h, followed by 24 h XIAP antisense or scrambled PMO treatments. Controls included untreated cells, vehicle (cells that were scraped in culture medium only). The cells that were treated with cytotoxic agent alone were also sham scrape loaded to account for any cell death due to the antisense delivery method.

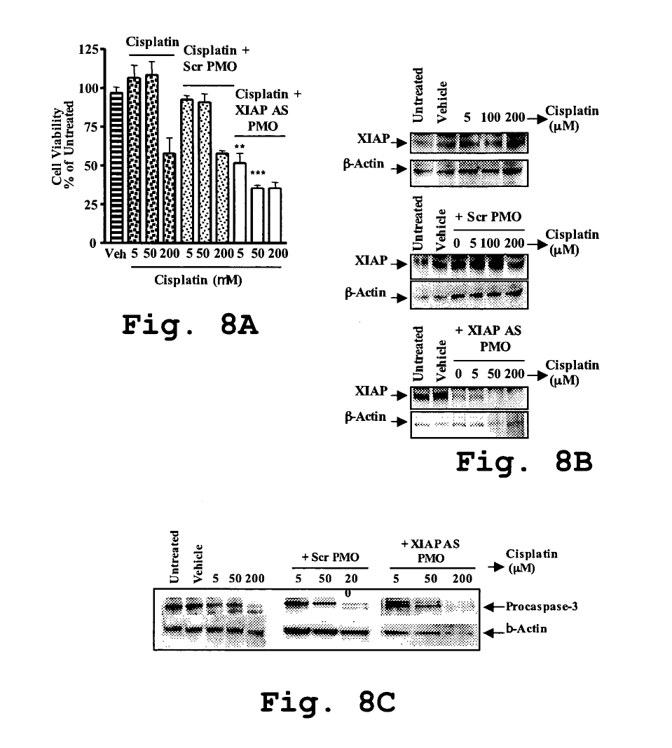

A significantly greater decrease in cell viability was observed in cells treated with a combination of XIAP antisense PMO+cisplatin compared to cisplatin alone or combination of scrambled PMO+cisplatin treatment groups (FIG. 8A). In the presence of XIAP antisense PMO, the data revealed that a several fold lower cisplatin concentration (5 μM) along with shorter duration of treatment (24 h) was required to induce a significant decrease in DU145 cell viability compared to cisplatin as a single agent (FIG. 1; resistance at 200□M, up to 72 h). Immunoblot analysis (FIGS. 8B and 8C) revealed a significant decrease in XIAP expression and procaspase-3 protein levels in the XIAP antisense PMO+cisplatin combination treatment group.

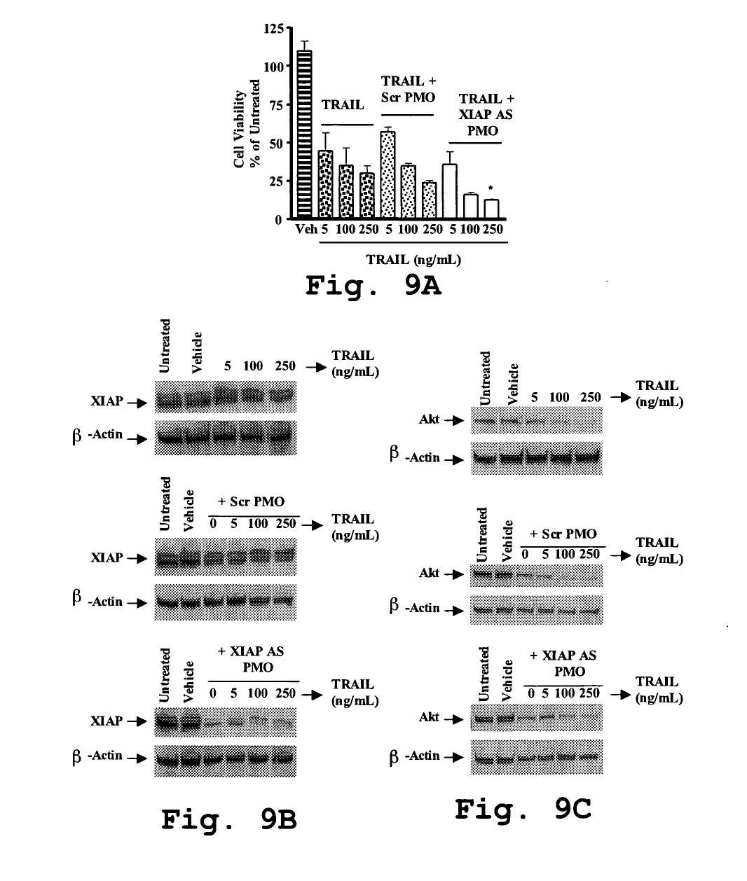

Another combination treatment schedule with TRAIL and XIAP antisense PMO revealed that treatment of DU145 cells with XIAP antisense PMO for 24 h followed by TRAIL treatment for another 24 h enhanced TRAIL sensitivity as seen with significantly increased cell death at 100-250 ng/ml concentrations in the XIAP antisense+TRAIL combination group compared to TRAIL alone or TRAIL+scrambled combination (FIG. 9A). Immunoblot analysis of the lysates revealed a marked decrease in XIAP levels in the XIAP antisense PMO alone and XIAP antisense+TRAIL combination treated cells. This decrease in XIAP levels was not observed in the TRAIL alone or TRAIL+scrambled PMO treated cells as shown in FIG. 9B. A corresponding decrease in Akt (FIG. 9C) was observed in the XIAP antisense, TRAIL and XIAP antisense+TRAIL combination treated cells as shown before in FIGS. 5C and 7C. These results demonstrate that prior downregulation of XIAP and Akt expression tends to enhance TRAIL-induced apoptosis in DU145 human prostate cancer cells.

### EXAMPLE 5

### XIAP Antisense PMO Potentiates Resistance to Ionizing Radiation

Radiation therapy is one of the treatment protocols highly desired for the treatment of majority of cancers. However, resistance to ionizing radiation remains a major hurdle in the treatment of cancer. The mechanism by which ionizing radiation causes cell death is traditionally thought to be the induction of stranded breaks in DNA as well as damage to the cell membrane, which could lead to the activation of downstream pathways that contribute to cell death.Ionizing radiation triggers apoptosis primarily via the release of cytochrome C from the mitochondria and subsequent activation of the initiator caspase (caspase-9) pathway. Furthermore, ionizing radiation-induced apoptosis is thought to cause activation of caspase-8. Therefore activation of programmed cell (apoptosis) seems to be the principal mode by which cancer cells die following exposure to ionizing radiation. IAPs family members regulate apoptosis by inhibiting members of the caspase family, making them the most downstream natural antiapoptotic factors. X-Linked inhibitor of apoptosis (XIAP) is considered the key and most potent caspase inhibitor. Translational upregulation of XIAP has been shown to occur following treatment with acute low dose ionizing radiation and this correlates with increased resistance to radiation (Holcik, Gibson et al. 2001). This Example demonstrates that manipulation of XIAP levels in cells using antisense PMO targeting XIAP expression will induce apoptosis after treatment with ionizing radiation.

XIAP antisense and scrambled PMOs were synthesized and purified at AVI BioPharma, Inc. (Corvallis, Oreg.) with purity greater than 95% as determined by reverse phase high-performance liquid chromatography (HPLC) and MALDI TOF mass spectroscopy. The base compositions of the XIAP antisense oligomers are 5′-CTG TTA AAA GTC ATC TTC TC-3′ (SEQ ID NO:7) and 5′-GGA CTT GTC CAC CTT TTC-3′ (SEQ ID NO:10). A sequence-scrambled control oligomer, 5′-CTT GAT AGA ATC TAC TCT CT-3′ (SEQ ID NO:13), was also used. The lyophilized PMOs are water-soluble and so were dissolved in sterile distilled water for in vitro experiments.

DU145 human prostate cancer cells were obtained from ATCC (Manassas, Va.). RPMI-1640 culture media was purchased from Hyclone Laboratories, Inc. Logan, Utah. Penicillin and streptomycin were obtained from Gibco, Grand Island, N.Y. The cells were routinely cultured in RPMI-1640 supplemented with 10% Fetal Bovine Serum (Hyclone Laboratories, Inc, Logan, Utah), 10 U/mL penicillin and 10 μg/mL streptomycin in a 5% CO₂ and 95% air humidified incubator at 37° C. For cell viability assays, cells were seeded at 500,000 cells/well in a 6-well plate (Becton Dickinson and Company, Franklin Lakes, N.J.) and allowed to reach at least 80% confluence. After treatment with gamma radiation, culture media was aspirated and cell viability was determined by trypan blue exclusion assay. In this assay cells were trypsinized and resuspended in media to achieve a homogenous mixture. An aliquot of cell suspension was then mixed with an equal volume of 0.4% trypan blue solution (Sigma, St. Louis, Mo.). Cells numbers in 10 μL of the resultant mixture were recorded using a hemocytometer.

Cells were treated with XIAP antisense or scrambled PMOs by scrape loading for 24 h and then followed by treatment with 10 Gy of gamma radiation. Antisense PMO was delivered at 37° C. whereas treatment with gamma radiation was done at room temperature in serum-containing culture media.

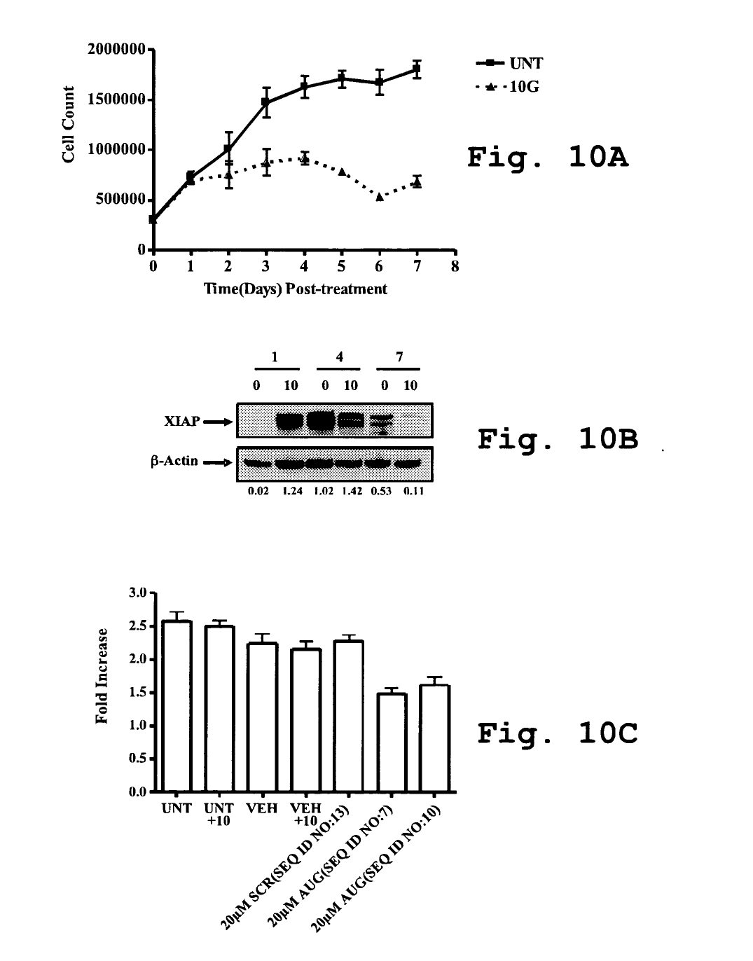

The effect of gamma radiation on DU145 and M12 cell viability was investigated by treating DU145 cells with 10 Gy of gamma radiation and cell viability was monitored by trypan blue exclusion test at different time points. As shown in FIG. 10A, the cells were resistant at 24 h following radiation treatment. This was correlated with increased XIAP levels, particularly at one day post irradiation, as shown in the western immunoblot (FIG. 10B). Cell growth was observed to decrease at later time points with a corresponding decline in XIAP levels in the treated population compared to the control. The data above suggests that gamma radiation sensitivity in DU145 cells is inversely related to XIAP expression. A combination treatment using XIAP antisense PMO and gamma radiation was conducted in a scheduled manner. In one case, cells were treated with XIAP antisense (SEQ ID NOS:7 and 10) or scrambled PMO (SEQ ID NO:13) for 24 h and then followed by 10 Gy of gamma radiation. A decrease in cell viability, expressed as “Fold Increase” was observed in cells treated with XIAP antisense PMO in combination with 10 Gy of radiation compared to radiation alone or in combination with scrambled PMO as shown in FIG. 10C. In FIG. 10C “Unt” refers to untreated cells, “Veh” are mock scrape loaded cells and all three PMO-treated data points were from cells treated with 10 Gy of ionizing radiation.

Sequence Listing

| | | |
|---|---|---|
| SEQ ID | GenBank | |
| NO. | Acc. No. | Location |

| | | | |
|---|---|---|---|
| Target Sequences | | | |
| gagaagatgacttttaacag | 1 | AL121601 | 13779-13798 |
| cctattttcaagagaagatg | 2 | AL121601 | 13768-13787 |
| cttttaacagttttgaagg | 3 | AL121601 | 13789-13807 |
| gaaaaggtggacaagtcc | 4 | AL121601 | 13752-13769 |
| gctggattttatgctttag | 5 | AL121601 | 14643-14661 |
| gtgaaggtgataaagtaaagtgc | 6 | AL121601 | 16741-16763 |
| Oligomer | | | |
| Targeting Sequences | | | |
| CTGTTAAAAGTCATCTTCTC | 7 | U45880 | 28-47 |
| CATCTTCTCTTGAAAATAGG | 8 | U45880 | 17-36 |
| CCTTCAAAACTGTTAAAAG | 9 | U45880 | 38-56 |
| GGACTTGTCCACCTTTTC | 10 | U45880 | 1-18 |
| CTAAAGCATAAAATCCAGC | 11 | U45880 | 892-910 |
| GCACTTTACTTTATCACCTTCAC | 12 | U45880 | 911-933 |
| CTTGATAGAATCTACTCTCT | 13 | N/A | N/A |
| TRAIL | | | |
| vrergpqrvaahitgtrgrsntl | 14 | NP003801 | 114-281 |
| sspnsknekalgrkinswessrs | | | |
| ghsflsnlhlrngelvihekgfy | | | |
| yiysqtyfrfqeeikentkndkq | | | |
| mvqyiykytsypdpillmksarn | | | |
| scwskdaeyglysiyqggifelk | | | |
| endrifvsvtnehlidmdheasf | | | |
| fgaflvg | | | |

1. A method of enhancing the lethality of an anti-cancer agent selected from radiation or a small-molecule chemotherapeutic compound on mammalian cancer cells, comprising
(a) exposing the cells, before, during or after exposing the cells to the anti-cancer agent, to an antisense compound characterized by:
(i) 12-40 morpholino subunits,
(ii) a substantially uncharged, phosphorus-containing backbone linking a morpholino nitrogen of one subunit to a 5′ exocyclic carbon of an adjacent subunit,
(iii) active uptake by mammalian cancer cells,
(iv) a base sequence that is complementary to a target region containing at least 12 contiguous bases in a preprocessed or processed human X-linked inhibitor of apoptosis protein (XIAP) and which includes at least 6 contiguous bases of the sequence selected from the group consisting of: SEQ ID NOS: 7-12 and
(v) capable of hybridizing with a processed or preprocessed human X-linked inhibitor of apoptosis protein (XIAP) transcript to form a heteroduplex structure having a Tm of dissociation of at least 45° C.,
in an amount of antisense compound effective to enhance the lethality/dose of the cells to the agent.

2. The method of claim 1, wherein the morpholino subunits in the antisense compound to which the cells are exposed are joined by phosphorodiamidate linkages, in accordance with the structure: 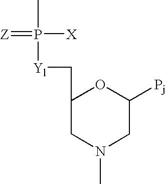
where Y₁═O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino.

3. The method of claim 2, wherein X═NR₂, where each R is independently hydrogen or methyl in the compound administered.

4. The method of claim 1, wherein the antisense compound to which the cells are exposed is effective to target the start site of the processed XIAP transcript, and which includes at least 6 contiguous bases of the sequence selected from the group consisting of: SEQ ID NOS:7-9.

5. The method of claim 4, wherein the antisense compound to which the cells are exposed includes a base sequence selected from the group consisting of: SEQ ID NOS:7-9.

6. The method of claim 1, wherein the antisense compound to which the cells are exposed is effective to target the IRES site of the processed XIAP transcript, and which includes at least 6 contiguous bases of the sequence identified as SEQ ID NO:10.

7. The method of claim 6, wherein the antisense compound to which the cells are exposed includes a base sequence identified as SEQ ID NO: 10.

8. The method of claim 1, wherein the antisense compound to which the cells are exposed is effective to target a splice site of the preprocessed XIAP transcript, and has a base sequence that is complementary to a target region containing at least 12 contiguous bases in a preprocessed XIAP transcript, and which includes at least 6 contiguous bases of the sequence selected from the group consisting of: SEQ ID NOS: 11 and 12.

9. The method of claim 8, wherein the antisense compound to which the cells are exposed includes a base sequence selected from the group consisting of: SEQ ID NOS:11 and 12.

10. The method of claim 1, which further includes, prior to said exposing, of identifying the cells as showing diminished responsiveness to the agent, as evidenced by on diminished lethality per dose toxic agent over time.

11. The method of claim 1, for use in treating a cancer in a mammalian subject, wherein said exposing includes administering said antisense compound to the subject before, during, or after treating the subject with said agent.

12. The method of claim 11, wherein the antisense compound and agent are administered alternately to the subject, at intervals separated by 1-5 days.

13. The method of claim 12, wherein the agent administered is cis-platin or an analog thereof.

14. The method of claim 11, wherein said cancer is an androgen-independent prostate cancer, wherein said exposing includes administering said antisense compound to the subject before, during, or after treating the subject with said agent.

15. A method of treating a cancer in a mammalian subject, comprising
(a) administering to the subject, an antisense compound characterized by:
(i) 12-40 subunits,
(ii) a substantially uncharged, phosphorus-containing backbone linking said subunits,
(iii) active uptake by mammalian cells,
(iv) a base sequence that is complementary to a target region containing at least 12 contiguous bases in a preprocessed or processed human X-linked inhibitor of apoptosis protein (XIAP) and which includes at least 6 contiguous bases of the sequence selected from the group consisting of: SEQ ID NOS: 7-12 and
(v) capable of hybridizing with a processed or preprocessed human X-linked inhibitor of apoptosis protein (XIAP) transcript to form a heteroduplex structure having a Tm of dissociation of at least 45° C., and (b) administering to the subject, a TRAIL protein characterized by the sequence SEQ ID NO:14, where
(i) the step (b) is carried out at least 6 hours after step (a),
(ii) the amount of oligonucleotide analog administered is effective to inhibit XIAP levels in the subject's cancer cells;
(iii) the amount of TRAIL administered is effective to inhibit growth of the cancer cells, and
(iv) the extent of inhibition of growth of the cancer cells by steps (a) and (b) alone is greater than by step (b) alone.

16. The method of claim 1, wherein the antisense compound to which the subject is exposed is composed of morpholino subunits joined by a substantially uncharged, phosphorus-containing backbone linking a morpholino nitrogen of one subunit to a 5′ exocyclic carbon of an adjacent subunit,

17. The method of claim 16, wherein the antisense compound to which the subject is exposed is formed with phosphorodiamidate linkages, in accordance with the structure: 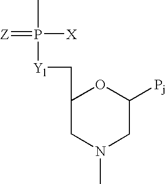
where Y₁═O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino.

18. The method of claim 17, wherein X═NR₂, where each R is independently hydrogen or methyl in the compound administered.

19. The method of claim 15, wherein the antisense compound to which the subject is exposed is effective to target the start site of the processed XIAP transcript, and which includes at least 6 contiguous bases of the sequence selected from the group consisting of: SEQ ID NOS:7-9.

20. The method of claim 19, wherein the antisense compound to which the subject is exposed includes a base sequence selected from the group consisting of: SEQ ID NOS:7-9.

21. The method of claim 15, wherein the antisense compound to which the subject is exposed is effective to target the IRES site of the processed XIAP transcript, and which includes at least 6 contiguous bases of the sequence identified as SEQ ID NO:10.

22. The method of claim 21, wherein the antisense compound to which the subject is exposed includes a base sequence identified as SEQ ID NO: 10.

23. The method of claim 15, wherein the antisense compound to which the subject is exposed is effective to target a splice site of the preprocessed XIAP transcript, and has a base sequence that is complementary to a target region containing at least 12 contiguous bases in a processed human androgen receptor transcript, and which includes at least 6 contiguous bases of the sequence selected from the group consisting of: SEQ ID NOS: 11 and 12.

24. The method of claim 23, wherein the antisense compound to which the subject is exposed includes a base sequence selected from the group consisting of: SEQ ID NOS:11 and 12.

25. The method of claim 15, for use in treating an androgen-independent prostate tumor, which further includes, prior to administering step (a) determining that the subject's prostate cancer is androgen-independent.

26. The method of claim 25, wherein said determining includes
(a) administering said antisense compound to the subject,
(b) at a selected time after said administering, obtaining a sample of a body fluid from the subject; and
(c) assaying the sample for the presence of a nuclease-resistant heteroduplex comprising the antisense compound complexed with a complementary-sequence portion of or preprocessed or processed XIAP transcript.

27. An oligonucleotide analog compound for use in cancer in a subject, characterized by:
(i) 12-40 morpholino subunits,
(ii) a substantially uncharged, phosphorus-containing backbone linking a morpholino nitrogen of one subunit to a 5′ exocyclic carbon of an adjacent subunit,
(iii) active uptake by mammalian cancer cells,
(iv) a base sequence that is complementary to a target region containing at least 12 contiguous bases in a preprocessed or processed human X-linked inhibitor of apoptosis protein (XIAP) and which includes at least 6 contiguous bases of the sequence selected from the group consisting of: SEQ ID NOS: 7-12 and
(v) capable of hybridizing with a processed or preprocessed human X-linked inhibitor of apoptosis protein (XIAP) transcript to form a heteroduplex structure having a Tm of dissociation of at least 45° C.

28. The compound of claim 27, wherein the morpholino subunits in the antisense compound to which the cells are exposed are joined by phosphorodiamidate linkages, in accordance with the structure: 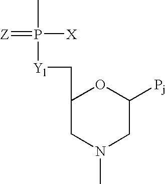
where Y₁═O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino.

29. The compound of claim 28, wherein X═NR₂, where each R is independently hydrogen or methyl in the compound administered.

30. The compound of claim 27, which is effective to target the start site of the processed XIAP transcript, and which includes at least 6 contiguous bases of the sequence selected from the group consisting of: SEQ ID NOS:7-9.

31. The compound of claim 30, which includes a base sequence selected from the group consisting of: SEQ ID NOS:7-9.

32. The compound of claim 27, which is effective to target the IRES site of the processed XIAP transcript, and which includes at least 6 contiguous bases of the sequence identified as SEQ ID NO:10.

33. The compound of claim 32, which includes a base sequence identified as SEQ ID NO: 10.

34. The compound of claim 27, which is effective to target a splice site of the preprocessed XIAP transcript, and has a base sequence that is complementary to a target region containing at least 12 contiguous bases in a processed human androgen receptor transcript, and which includes at least 6 contiguous bases of the sequence selected from the group consisting of: SEQ ID NOS: 11 and 12.

35. The compound of claim 34, which includes a base sequence selected from the group consisting of: SEQ ID NOS:11 and 12.


SEQUENCE LISTING (14 sequences)

SEQ ID NO: 1 - DNA, 20 bp, Homo sapiens
gagaagatgacttttaacag

SEQ ID NO: 2 - DNA, 20 bp, Homo sapiens
cctattttcaagagaagatg

SEQ ID NO: 3 - DNA, 19 bp, Homo sapiens
cttttaacagttttgaagg

SEQ ID NO: 4 - DNA, 18 bp, Homo sapiens
gaaaaggtggacaagtcc

SEQ ID NO: 5 - DNA, 19 bp, Homo sapiens
gctggattttatgctttag

SEQ ID NO: 6 - DNA, 23 bp, Homo sapiens
gtgaaggtgataaagtaaagtgc

SEQ ID NO: 7 - DNA, 20 bp, Artificial sequence
  antisense oligomer sequence complementary to nucleotides 28-47 of human XIAP gene
ctgttaaaagtcatcttctc

SEQ ID NO: 8 - DNA, 20 bp, Artificial sequence
  antisense oligomer sequence complementary to nucleotides 17-36 of human XIAP gene
catcttctcttgaaaatagg

SEQ ID NO: 9 - DNA, 19 bp, Artificial sequence
  antisense oligomer sequence complementary to nucleotides 38-56 of human XIAP gene
ccttcaaaactgttaaaag

SEQ ID NO: 10 - DNA, 18 bp, Artificial sequence
  antisense oligomer sequence complementary to nucleotides 1-18 of human XIAP gene
ggacttgtccaccttttc

SEQ ID NO: 11 - DNA, 19 bp, Artificial sequence
  antisense oligomer sequence complementary to nucleotides 892-910 of human XIAP gene
ctaaagcataaaatccagc

SEQ ID NO: 12 - DNA, 23 bp, Artificial sequence
  antisense oligomer sequence complementary to nucleotides 911-933 of human XIAP gene
gcactttactttatcaccttcac

SEQ ID NO: 13 - DNA, 20 bp, Artificial sequence
  a nonsense/scrambled antisense oligomer sequence
cttgatagaatctactctct

SEQ ID NO: 14 - protein, 168 aa, Homo sapiens
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG